(19) United States
(12) Patent Application Publication
(10) Pub. No.: US 2026/0229355 A1
(43) Pub. Date: Aug. 6, 2026

(54) SYSTEM AND METHOD FOR DECONVOLUTION OF BREAST TISSUE AND BREAST MILK CELL PROPORTIONS USING REFERENCE DNA METHYLATION PROFILES

(71) Applicant: The Trustees of Dartmouth College, Hanover, NH (US)

(72) Inventors: Brock C. Christensen, Lebanon, NH (US); Meghan E. Muse, Hanover, NH (US); Lucas A. Salas, Lebanon, NH (US)

(21) Appl. No.: 19/149,538

(22) PCT Filed: Jan. 19, 2024
(86) PCT No.: PCT/US24/12165

§ 371 Date: Jul. 19, 2025

Related U.S. Application Data
(60) Provisional application No. 63/440,312, filed on Jan. 20, 2023.

(51) Int. Cl. G16H 50/20; G16B 35/20; G16H 20/10; G16H 40/67; G16H 50/70

(52) U.S. Cl. CPC G16H 50/20; G16B 35/20; G16H 20/10; G16H 40/67; G16H 50/70

(57) ABSTRACT

This invention provides a system and method for use of a library for reference-based deconvolution of breast tissue and/or breast milk DNA methylation data assayed using the Illumina Infinium MethylationEPIC BeadChip. A library is accessed, containing information related to an estimate of relative proportions of epithelial cells, endothelial cells, fibroblasts, adipocytes, B cells, CD4+ T cells, CD8+ T cells, NK cells, monocytes, and neutrophils associated with the breast tissue and/or breast milk. The library is applied to diagnosis and treatment of a medical condition based upon patient information obtained from a sample of patient breast tissue and/or breast milk.

### RELATED APPLICATION

This application claims the benefit of co-pending U.S. Provisional Application Ser. No. 63/440,312, entitled SYSTEM AND METHOD FOR DECONVOLUTION OF BREAST TISSUE AND BREAST MILK CELL PROPORTIONS USING REFERENCE DNA METHYLATION PROFILES, filed Jan. 20, 2023, the teachings of which are expressly incorporated herein by reference.

### STATEMENT REGARDING FEDERALLY SPONSORED RESEARCH OR DEVELOPMENT

This invention was made with U.S. government support under Grant Numbers P20GM104416 and UH3OD023275, awarded by the National Institutes of Health (NIH). The government has certain rights in this invention.

### FIELD OF THE INVENTION

This invention relates to cell-type libraries for use in the analysis and diagnosis of various diseases and/or cancers.

### BACKGROUND OF THE INVENTION

Breast cancer presents a major public health burden with an estimated 281,550 new cases and 43,600 deaths in 2021 in women in the United States alone. See Bibliography references item (1). The high burden of disease and the increased survival observed when diagnoses are made at earlier stages of disease (1) underscore the importance of early detection in breast cancer. Precision prevention strategies are limited for breast cancer and assessment of tissue-specific molecular profiles offer new opportunities understand and act on individual breast cancer risk.

Documented intrinsic factors associated with breast cancer risk include age, family history of disease, and genetic factors such as high penetrance germline mutations in BRCA1 and BRCA2 and low penetrance SNPs (Bibliography reference items 2-7). While genetic approaches have aided in identifying women at risk of developing breast cancer (8), they provide an incomplete picture of disease risk. The study of epigenetic alterations related to disease risk, such as alterations to DNA methylation, presents an opportunity to expand upon our molecular understanding of disease risk and lifestyle-associated risk factors. Lifestyle factors associated with breast cancer risk include alcohol consumption, diet, and reproductive factors such as parity (2, 3). Other reproductive factors, such as earlier age at menarche and later age at first birth also have been associated with increased breast cancer risk (9, 10).

DNA-based deconvolution allows for identification and quantification of cell types in bio specimens that are complex mixtures of cells without the need to preserve cell membranes. By way of background, commonly assigned U.S. patent application Ser. No. 17/670,346, entitled ENHANCED DNA METHYLATION LIBRARY FOR DECONVOLUTING PERIPHERAL BLOOD, filed Feb. 11, 2022, the teaching of which are incorporated by reference, teaches use of deconvolution techniques to analyze and diagnose blood disorders

Early in breast carcinogenesis, alterations to DNA methylation are vast (11). The majority of the scope of DNA methylation alterations are already present in pre-invasive ductal carcinoma in situ (DCIS) relative to invasive tumors (12,13), indicating that alterations to DNA methylation occur early in breast carcinogenesis. Additionally, it has been demonstrated that histologically normal tissue collected from the same breast as a tumor harbors alterations to DNA methylation relative to breast tissue collected from the opposite breast (14), suggesting that early alterations to DNA methylation are present in a field of cells in the affected breast.

Prior work in women without breast cancer demonstrated that DNA methylation profiles in solid breast tissue are associated with age, an established breast cancer risk factor (15). As DNA methylation in the breast tissue of disease-free women is associated with established risk factors, and alterations to DNA methylation are known to occur early on in neoplastic transformation, DNA methylation profiles of breast tissue hold the potential to act as powerful biomarkers of disease risk. However, DNA methylation patterning is cell and tissue-specific, and maximizing the potential of DNA methylation measures in risk assessment and precision prevention requires measures in the target tissue. Of course, obtaining tissue with a breast biopsy is invasive and inappropriate for risk assessment.

However, breast milk contains shed breast epithelial cells (and other cell types), providing a tissue-specific biospecimen obtained noninvasively. Prospectively collected breast milk samples have shown that showed DNA methylation alterations in breast milk are associated with breast cancer risk (16).

### SUMMARY OF THE INVENTION

This invention overcomes disadvantages of the prior art by providing a novel library, and system and method for use therefor for the estimation of the cellular composition of breast tissue and breast milk and subsequent assessment of cell type-dependent associations with breast cancer and other breast diseases. More particularly, the system and method applies a novel reference-based cell type library to identify cellular composition-independent DNA methylation alterations associated with established breast cancer risk factors and assess whether these alterations are shared between solid breast tissue and breast milk in disease-free subjects.

In an illustrative embodiment, a system and method is provided for use of a library for reference-based deconvolution of breast tissue and/or breast milk DNA methylation data assayed using a methylation process. A library is accessed with a processor assembly, containing information related to an estimate of relative proportions of epithelial cells, endothelial cells, fibroblasts, adipocytes, B cells, CD4+ T cells, CD8+ T cells, NK cells, monocytes, and neutrophils associated with the breast tissue and/or breast milk. The library is applied by the processor assembly to diagnosis and treatment of a medical condition based upon patient information obtained from a sample of patient breast tissue and/or breast milk. Illustratively, the processor assembly provides information to a user interface based upon user requests thereto. The processor assembly can compare the relative proportions of epithelial cells, endothelial cells, fibroblasts, adipocytes, B cells, CD4+ T cells, CD8+ T cells, NK cells, monocytes, and neutrophils associated with the breast tissue and/or breast milk in a patient being diagnosed to relative proportions of one or more of epithelial cells, endothelial cells, fibroblasts, adipocytes, B cells, CD4+ T cells, CD8+ T cells, NK cells, monocytes, and neutrophils associated with the breast tissue and/or breast milk in a disease-free subject. Illustratively, the methylation process comprises Illumina Infinium MethylationEPIC BeadChip. The processor assembly can be associated with a computing system having a network-based communication arrangement between a user, a storage site for the library and a server assembly that accesses the library and performs the step of comparing. The processor can operate a non-transitory computer-readable medium for performing the system and method.

### BRIEF DESCRIPTION OF THE DRAWINGS

The invention description below refers to the accompanying drawings, of which:

FIG. 1A shows breast-specific DNA methylation reference library for cell type deconvolution in breast tissue and breast milk in a heatmap of the CpG weights assigned to each cell type for the 677 probes included in the breast-specific reference library. Mammary epithelial cells, endothelial cells, fibroblasts, adipocytes, B cells, CD4+ T cells, CD8+ T cells, NK cells, monocytes, and neutrophils were included in the library, wherein unsupervised hierarchical clustering of CpG loci and cell types, respectively, was conducted utilizing Manhattan distance;

FIG. 1B shows a distribution of the estimated relative proportion of each cell type represented in the reference library across solid breast tissue (n=95) and breast milk (n=48) samples;

FIGS. 2A and 2B shows CpG sites significantly differentially methylated with parity from epigenome-wide association analysis in solid breast tissue (parous relative to nulliparous), and in breast milk (parous prior to pregnancy relative to first time mothers) adjusted for cell type, in which dashed lines indicate a significance threshold of FDR<0.05;

FIG. 2C shows the overlap of identified hypermethylated and hypomethylated loci (P<0.01) in solid breast tissue and breast milk;

FIG. 2D shows enrichment analysis of all overlapping hypermethylated CpG loci (P<0.01) in solid breast tissue and breast milk;

FIGS. 3A and 3B show epigenome-wide association analysis identifying CpG sites that are significantly differentially methylated with increasing age, adjusted for estimated proportions of epithelial cells, endothelial cells, fibroblasts, adipocytes, monocytes, and neutrophils in solid breast tissue and breast milk, in which dashed lines indicate a significance threshold of FDR<0.05;

FIG. 3C shows overlap of identified hypermethylated and hypomethylated loci (P<0.01) with increasing age in solid breast tissue and breast milk;

FIG. 3D shows enrichment analysis of 772 overlapping hypermethylated CpG loci (P<0.01) with increasing age in solid breast tissue and breast milk;

FIGS. 4A and B show Association between age and DNA methylation in an independent data set of solid breast tissue (n=121, GSE101961) in (A) 130 shared hypomethylated loci (P<0.01, FIG. 3C) with increasing age in solid breast tissue and breast milk, and 772 shared hypermethylated loci (P<0.01, FIG. 3C) with increasing age in solid breast tissue and breast milk. Red loci indicate loci with consistent hypomethylation in (FIG. 4A) and hypermethylation in (FIG. 4B), respectively;

FIG. 4C shows differential methylation of CpG islands, assessed as the mean methylation of all loci mapping to a given CpG island, in TCGA tumor relative to adjacent normal tissue for the 223 CpG islands whose shores contain significantly hypermethylated CpG loci with increasing age in both solid breast tissue and breast milk;

FIG. 5A shows SFRP2 promoter CpG island shore DNA methylation in solid breast tissue, breast milk, and breast tumor tissue for unadjusted association between age and mean DNA methylation of the 10 overlapping hypermethylated loci SFRP2 (Table 2) in solid breast tissue and breast milk, respectively;

FIG. 5B shows a distribution of the mean methylation of the 10 overlapping hypermethylated loci in SFRP2 (Table 2) in tumor and adjacent normal TCGA samples across tumor subtypes;

FIG. 5C shows distribution of methylation of probes across the SFRP2 promoter in TCGA breast tumor and adjacent normal tissue, wherein Loci tracking to the promoter CpG island are indicated in black and loci tracking to the adjacent CpG island shore are indicated in grey Loci that are hypermethylated with increasing age in both solid breast tissue and breast milk are indicated in black;

FIG. 6 shows correlation between estimated relative proportions of immune cells and true relative proportions in the in silico mixtures (n=12), wherein component cell types include B cells, CD4+ T cells, CD8+ T cells, monocytes, neutrophils, and NK cells;

FIG. 7 shows estimated relative proportions of component cell types in the breast-specific reference library among an independent data set of isolated adipocytes from abdominal subcutaneous fat samples (n=30, GSE58622);

FIG. 8 shows correlation between subject age (years) and estimated relative proportions of cells in solid breast tissue (n=95), wherein component cell types include epithelial cells, endothelial cells, fibroblasts, adipocytes, B cells, CD4+ T cells, CD8+ T cells, monocytes, neutrophils, and NK cells;

FIG. 9 shows correlation between subject BMI (kg/m2) and estimated relative proportions of cells in solid breast tissue (n=95), wherein component cell types include epithelial cells, endothelial cells, fibroblasts, adipocytes, B cells, CD4+ T cells, CD8+ T cells, monocytes, neutrophils, and NK cells;

FIG. 10 shows correlation between subject reproductive age (years) and estimated relative proportions of cells in solid breast tissue (n=95), wherein component cell types include epithelial cells, endothelial cells, fibroblasts, adipocytes, B cells, CD4+ T cells, CD8+ T cells, monocytes, neutrophils, and NK cells;

FIGS. 11A and 11B show a distribution of the estimated relative proportion of each component cell type in solid breast tissue (n=95), and breast milk (n=48) by subject parity. Component cell types include epithelial cells, endothelial cells, fibroblasts, adipocytes, B cells, CD4+ T cells, CD8+ T cells, monocytes, neutrophils, and NK cells, wherein Bonferroni-corrected P values shown from two sample t-tests;

FIGS. 12A and 12B show a distribution of the estimated relative proportion of each component cell type in solid breast tissue (n=95), and breast milk (n=48) by subject family history of breast cancer. Component cell types include epithelial cells, endothelial cells, fibroblasts, adipocytes, B cells, CD4+ T cells, CD8+ T cells, monocytes, neutrophils, and NK cells, wherein Bonferroni-corrected P values shown from two sample t-tests;

FIG. 13 shows correlation between subject age (years) and estimated relative proportions of cells in breast milk (n=48), wherein component cell types include epithelial cells, endothelial cells, fibroblasts, adipocytes, B cells, CD4+ T cells, CD8+ T cells, monocytes, neutrophils, and NK cells;

FIG. 14 shows graphs with correlation between subject BMI (kg/m2) and estimated relative proportions of cells in breast milk (n=48), wherein component cell types include epithelial cells, endothelial cells, fibroblasts, adipocytes, B cells, CD4+ T cells, CD8+ T cells, monocytes, neutrophils, and NK cells;

FIG. 15 shows graphs with correlation between subject reproductive age (years) and estimated relative proportions of cells in breast milk (n=48), wherein component cell types include epithelial cells, endothelial cells, fibroblasts, adipocytes, B cells, CD4+ T cells, CD8+ T cells, monocytes, neutrophils, and NK cells;

FIGS. 16A-16D respectively show Epigenome-wide association analysis identifying CpG sites that are significantly differentially methylated in breast milk relative to solid breast tissue, adjusting only for age and parity, adjusting for age, parity, and estimated proportions of epithelial cells, endothelial cells, fibroblasts, adipocytes, monocytes, and neutrophils, adjusting for age, parity, and family history of disease, and adjusting for age, parity, family history of disease, and estimated proportions of epithelial cells, endothelial cells, fibroblasts, adipocytes, monocytes, and neutrophils. Red dashed lines indicate a significance threshold of FDR<0.05;

FIG. 16E shows an enrichment analysis for differentially methylated loci (FDR<0.05) in breast milk relative to solid breast tissue after adjustment for age, parity, and cellular composition;

FIGS. 17A and 17B show Epigenome-wide association analysis identifying CpG sites that are significantly differentially methylated with increasing reproductive age in solid breast tissue, and breast milk, adjusted for estimated proportions of epithelial cells, endothelial cells, fibroblasts, adipocytes, monocytes, and neutrophils, wherein dashed lines indicate a significance threshold of FDR<0.05;

FIG. 17C shows overlap of identified hypermethylated and hypomethylated loci (P<0.01) in solid breast tissue and breast milk;

FIGS. 18A and 18B show Epigenome-wide association analysis identifying CpG sites that are significantly differentially methylated in women with a family history of breast cancer relative to women without a family history of breast cancer in solid breast tissue, and breast milk, adjusted for estimated proportions of epithelial cells, endothelial cells, fibroblasts, adipocytes, monocytes, and neutrophils;

FIG. 18C shows overlap of identified hypermethylated and hypomethylated loci (P<0.01) in solid breast tissue and breast milk;

FIGS. 19A and 19B show Epigenome-wide association analysis identifying CpG sites that are significantly differentially methylated with increasing BMI in solid breast tissue, and pre-pregnancy BMI in breast milk, adjusted for estimated proportions of epithelial cells, endothelial cells, fibroblasts, adipocytes, monocytes, and neutrophils;

FIG. 19C shows overlap of identified hypermethylated and hypomethylated loci (P<0.01) in solid breast tissue and breast milk; and

FIG. 20 shows a generalized computer processor system arrangement for performing the steps of the method of the illustrative embodiment.

### DETAILED DESCRIPTION

The system and method herein is arranged based upon studies described below. The data of such studies is accessed by networked and/or local computing devices, and is employed by a computing processor arrangement that employs a non-transitory computer readable medium of program instructions to deliver analysis and diagnostic data relative to patients based upon DNA samples therefrom.

### Study Populations

Approximately half of subjects donating solid breast tissue (n=95; Susan G. Komen Tissue Bank) had a first degree family member with a history of breast or ovarian cancer (subsequently referred to as a family history of disease) compared to approximately a third of subjects donating breast milk (n=24; New Hampshire Birth Cohort Study; See Table 1 below).

TABLE 1
Subject demographics for two study cohorts

| | Solid Breast Tissue | | Breast Milk | | |
|---|---|---|---|---|---|
| | (n = 95) | | (n = 24) | | P Value |
| Age mean (SD) | 36.51 | (13.23) | 32.57 | (5.42) | 0.157 |
| Parity n (%) | | | | | <0.001 |
| 0 | 46 | (48.9) | 0 | (0.0) | |
| 1 | 14 | (14.9) | 10 | (41.7) | |
| 2 | 16 | (17.0) | 9 | (37.5) | |
| 3+ | 18 | (19.1) | 5 | (20.8) | |
| Reproductive Age mean | 13.74 | (6.31) | 16.84 | (5.95) | 0.143 |
| (SD) | | | | | |
| Family History of | 44 | (51.8) | 8 | (34.8) | 0.226 |
| Disease n (%) | | | | | |

Solid breast tissue donors were also, on average, slightly older and captured a wider range of ages with a mean age of 36.5 compared to breast milk donors with a mean age of 32.6. Additionally, while approximately half of solid breast tissue donors had never given birth, all breast milk donors had at least one child with 41.7% of samples being collected from mothers for which this was their first child.

### Normal Breast Tissues

Fresh frozen breast tissue was obtained from the Susan G. Komen Tissue Bank for 95 cancer-free women who donated breast biopsies. Briefly, samples classified as having a high proportion of epithelial cells by the Susan G Komen Tissue Bank study pathologist were selected from subjects with a wide age range and an approximately equal distribution of parous and nulliparous women. Following DNA extraction and bisulfite modification, DNA methylation is measured with the Illumina 450 k array as previously described. See Bibliography reference item (15) below. DNA methylation data are available at GSE88883.

### Breast Milk Samples

Bilateral breast milk samples (n=48) were obtained from participants (n=24) in the New Hampshire Birth Cohort Study (NHBCS). Characteristics of the NHBCS have been previously described (17). Briefly, pregnant women were enrolled between approximately 24 and 28 weeks gestation from prenatal clinics in New Hampshire. Eligibility criteria included age of 18-45 years, English literacy, the use of a private, unregulated water system (e.g., private well) at home, not planning to move, and singleton pregnancies. Data were obtained from questionnaires and medical record reviews including subject characteristics, lifestyle factors, reproductive history, and general health. Participants were asked to bring bilateral breast milk specimens to the postpartum follow-up appointment, collected approximately 6 weeks postpartum. Breast milk was processed, DNA extracted and bisulfite modified, and DNA methylation was measured with the Illumina 450 k array as previously described (16). DNA methylation data are available at GSE133918

### Data Processing

Sample intensity data (IDAT) files for solid breast tissue from GEO data set GSE88883 (15) and for breast milk samples from GEO data set GSE133918 (16). IDAT files were processed using the functional normalization (funnorm) method from the R package min/i (18) and subsequent probe-type normalization was completed using beta-mixture quantile normalization (BMIQ) (19). Probes with a detection P value>1.0×10-6 in more than 5% of samples in either dataset, CpGs with common SNP(s) in the probe that are within 5 base pairs of the target CpG dinucleotide, probes previously described to be potentially cross-hybridizing (20), and sex-specific probes were excluded from subsequent analysis. Of the 485,512 probes included on the Infinium HumanMethylation450 BeadChip, 414.950 were included in the final analysis.

### Reference Library Development and Cell-Type Estimation

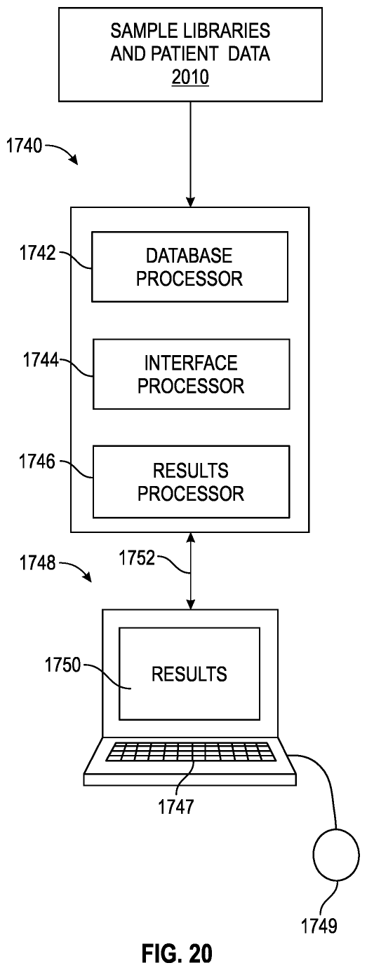

Among cell types present in breast tissue and breast milk, cell-specific differential methylation is identified and developed a novel reference library for DNA methylation-based cell type deconvolution. Data for mammary epithelial (n=2), endothelial (n=2), and fibroblast (n=2) cell lines (GSE74877) (21) is used, with isolated adipocytes from abdominal subcutaneous fat (n=29) in obese and non-obese women (GSE67024) (22), and purified B cells (n=6), CD4+ T cells (n=7), CD8+ T cells (n=6), NK cells (n=6), monocytes (n=6), and neutrophils (n=6; GSE110554) (23). CpG loci that were included in the above datasets were restricted to those that passed all filtering steps for both the solid breast tissue and breast milk data as previously described under data processing. The IDOL computer software algorithm operating in the below-described computing environment (FIG. 20) is used to identify the optimal reference library for each cell type (24).

To validate the identified reference library, the system and method utilizes DNA methylation profiles from in silico mixtures of immune cells (n=12; GSE110554) (23), and isolated adipocytes from abdominal subcutaneous fat (n=30; GSE58622). Relative proportions of all cell types were estimated using the epidish function (25) and specifying the robust partial correlations (RPC) method (26). Library performance was evaluated using Pearson correlation tests between the true relative proportions of cells present in in silico mixtures and estimated proportions from the novel reference library (which is typically electronically stored and accessible over a network environment) herein. Relative proportions of reference library cell types were estimated using DNA methylation data from solid breast tissue (n=95) and breast milk (n=48) applying the identified reference library and the RPC method within the epidish function.

### Statistical Analysis

Interrogated risk factors for analyses included age (continuous), parity (binary), reproductive age (continuous), BMI (continuous), and family history of breast or ovarian cancer (binary). Reproductive age was calculated as:

$$Reproductive Age = Age at First Birth - Age at Menarche women with at least one live birth , and$$

$$Reproductive Age = Age at Sample Collection - Age at Menarche for nulliparous women .$$

Univariate analyses comparing estimated cellular proportions of solid breast tissue and breast milk, respectively, and continuous breast cancer risk factors (age, reproductive age, and BMI) were conducted using a Pearson correlation coefficient using the R package stats. Analyses comparing estimated cellular proportions of solid breast tissue and breast milk, respectively, and binary breast cancer risk factors (parity and family history of breast or ovarian cancer) were conducted using two-sample t-tests with Bonferroni correction from the R package rstatix.

Differential methylation was assessed using linear mixed-effects models, modeling the relationship between logit-transformed beta values (M values) and breast cancer risk factors. Models were also adjusted for the estimated relative proportions of epithelial cells, endothelial cells, fibroblasts, adipocytes, monocytes, and neutrophils.

The annotation data for the Illumina HumanMethylation450 array was used to define the genomic context of included CpG loci. Cochran-Mantel-Haenszel tests were used to assess the enrichment of CpG island context among differentially methylated CpG loci (P<0.01) in both solid breast tissue and breast milk, separately for differentially hypermethylated and differentially hypomethylated CpG sets. Cochran-Mantel-Haenszel tests allow adjustment for array probe type, and the denominator was all 414,950 CpG loci included in the analysis.

### Data for Validation Analysis and Relevance to Tumor

To validate age-related differentially methylated (P<0.01) CpG loci shared between biospecimen types, data obtained from normal breast tissue in an independent and publicly available dataset (n=121. GSE101961) (27) is leveraged. Data were processed and cell-type proportions were estimated as described above.

DNA methylation data is obtained from 392 breast tumors and 82 adjacent normal samples from The Cancer Genome Atlas (TCGA) (11). Data were normalized as described above. The mean methylation status of CpG islands with identified hypermethylated shores with increasing age in solid breast tissue and breast milk was calculated across all CpG loci mapping to each island.

### Data Availability

All data used in this study are publicly available and can be accessed from GEO under the accession numbers GSE88883 (solid tissue samples) and GSE133918 (breast milk samples).

### Results

### Study Populations

DNA methylation is measured with (e.g.) the Illumina 450 k array in solid breast tissue samples from 95 healthy donors to the Susan G. Komen Tissue Bank. See Table 1 above. Of these individuals, 46 (48.9%) were nulliparous and the mean age was 36.5 years old. DNA methylation also was measured with the Illumina 450 k array in 48 paired breast milk samples (left and right breast) approximately 6 weeks postpartum from 24 lactating mothers in the New Hampshire Birth Cohort Study (Table 1). The mean age for breast milk donors was 32.6 years. This was the first live birth for 10 (41.7%) of the included mothers.

Cellular Composition of Solid Breast Tissue and Breast Milk Estimated with DNA Methylation

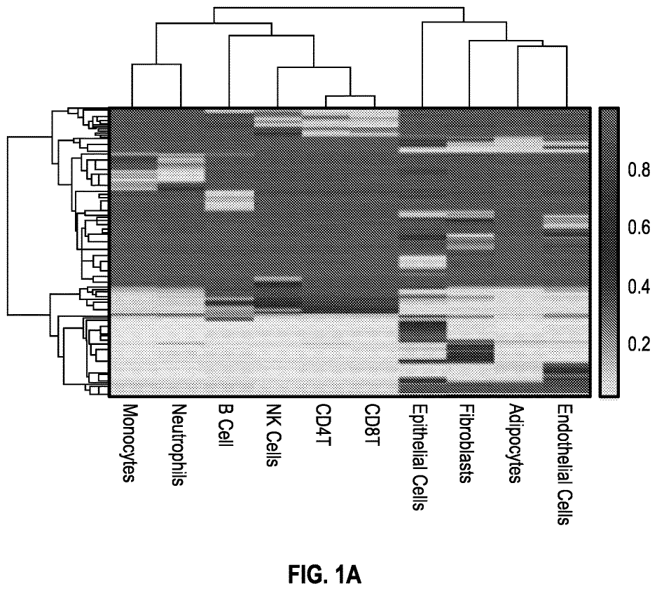

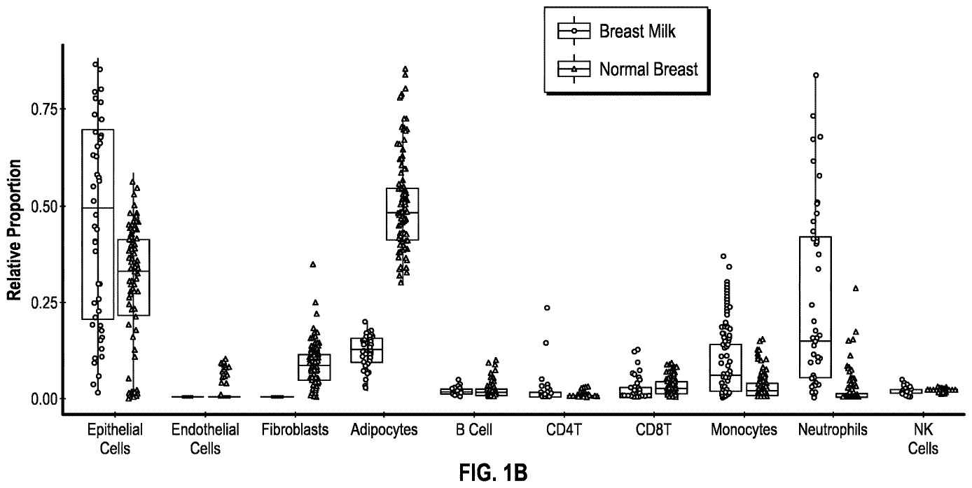

The system and method produces a novel reference library for DNA methylation-based cell type deconvolution in breast milk and solid breast tissue using publicly available data for mammary epithelial, endothelial, and fibroblast cell lines (GSE74877), isolated adipocytes from abdominal subcutaneous fat in obese and non-obese women (GSE67024), and purified B cells, CD4+ T cells, CD8+ T cells, NK cells, monocytes, and neutrophils (GSE110554). The optimized reference library included 677 probes to estimate the relative proportions of epithelial cells, endothelial cells, fibroblasts, adipocytes, B cells. CD4+ T cells, CD8+ T cells, NK cells, monocytes, and neutrophils (FIG. 1A).

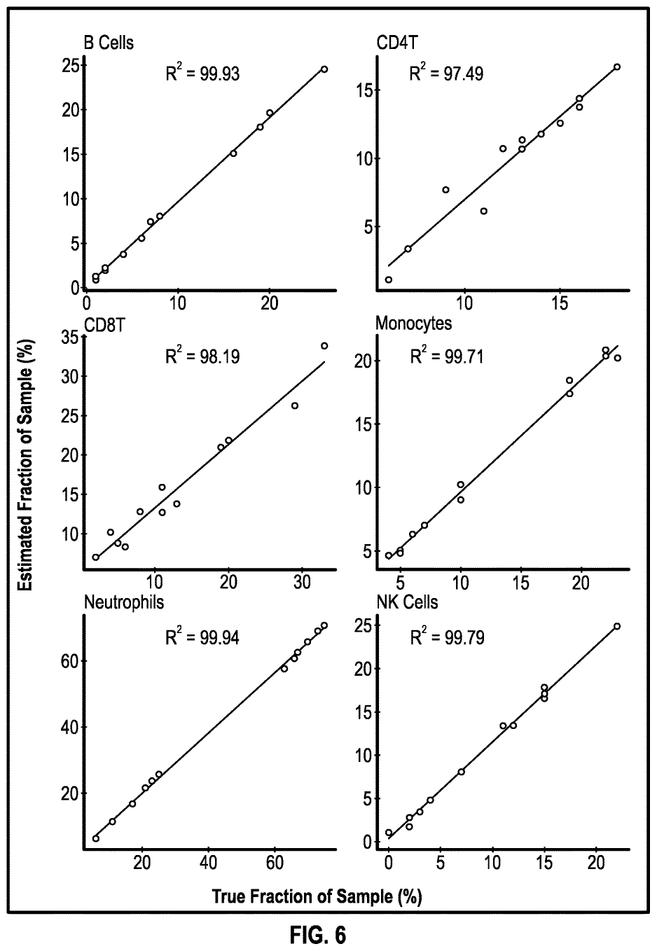

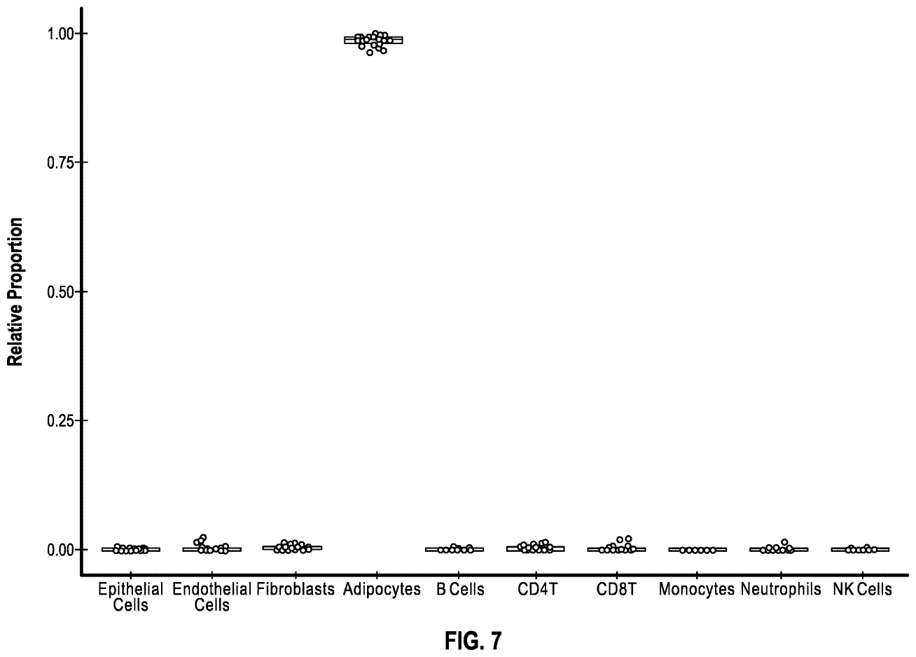

The accuracy and performance of the breast-specific reference library is then validated herein. First, the library is applied to estimate relative proportions of immune cells from in silico mixtures of data derived from purified cell type DNA methylation profiles (n=12). All six immune cell types demonstrated strong correlation (R2>0.97) with the true relative proportions in the in silico mixtures (FIG. 6). Then, in an independent data set of isolated adipocytes from abdominal subcutaneous fat (n=30; GSE58622), the breast-specific reference library estimated that the median adipocyte content of interrogated samples was 99% (FIG. 7).

Applying the library to breast tissue and breast milk DNA methylation data, expected differences in the cellular composition of solid breast tissue and breast milk (FIG. 1B) are observed. Among the 95 solid breast tissue samples, the cell types with the highest median estimated relative abundance were adipocytes (48.2%), epithelial cells (33.0%), and fibroblasts (8.3%). Among the 48 breast milk samples, the most abundant cell types were epithelial cells (49.5%), neutrophils (15.0%), and adipocytes (12.6%).

Associations of Biospecimen Cellular Composition with Breast Cancer Risk Factors

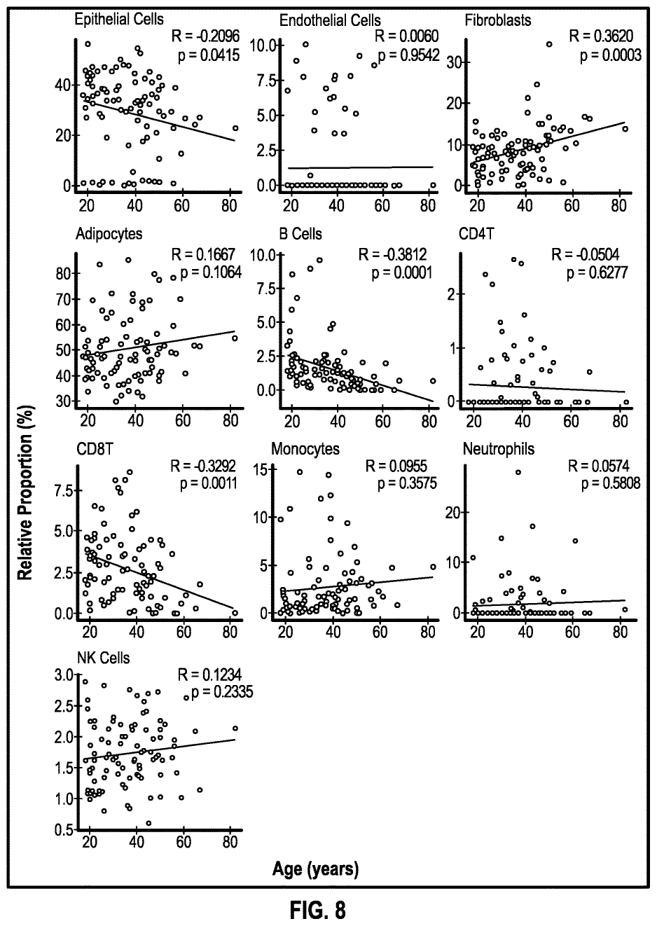

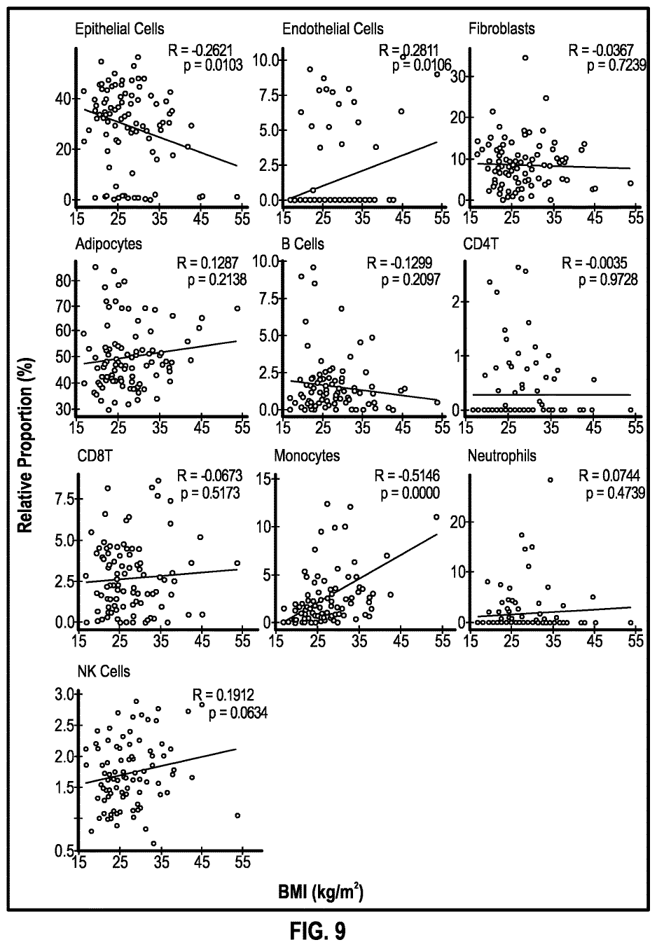

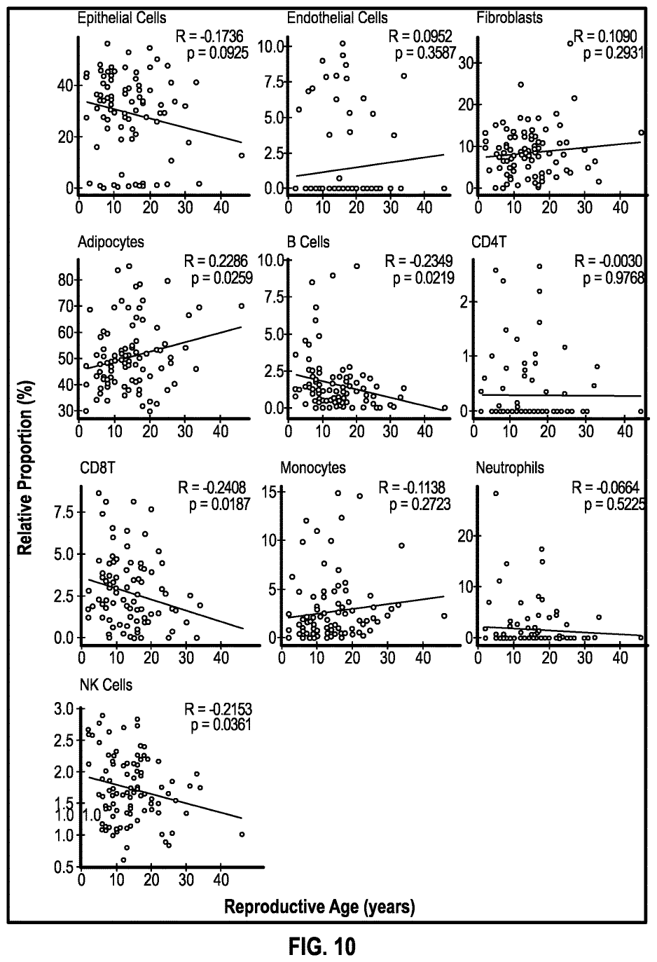

Breast cancer risk factors are associated with sample cellular composition in solid breast tissue. Age was significantly negatively associated with the relative proportion of epithelial cells, B cells, and CD8+ T cells (P<0.05), and significantly positively associated with fibroblast proportions (P<0.05, FIG. 8). BMI was significantly negatively associated with epithelial cell proportions (P<0.05) and significantly positively associated with endothelial cell and monocyte proportions (P<0.05, FIG. 9). Reproductive age was significantly negatively associated with B cell, CD8+ T, and NK cell proportions (P<0.05), and significantly positively associated with adipocyte proportions (P<0.05, FIG. 10).

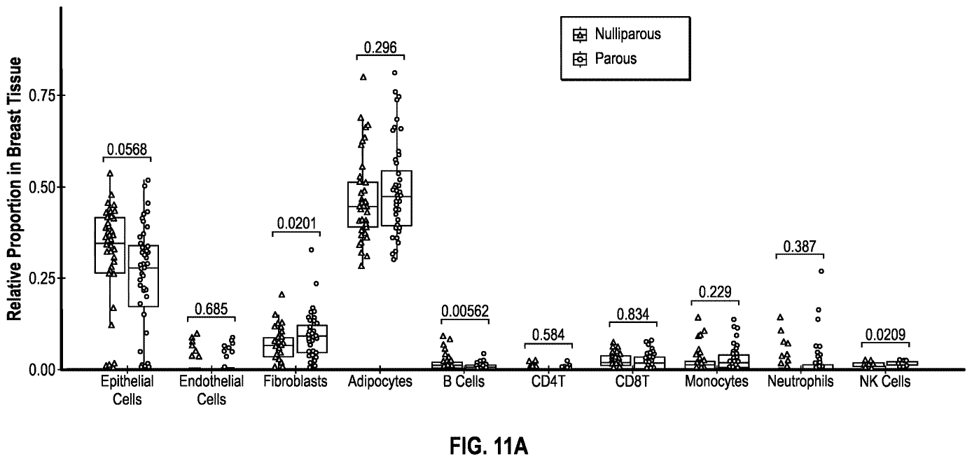

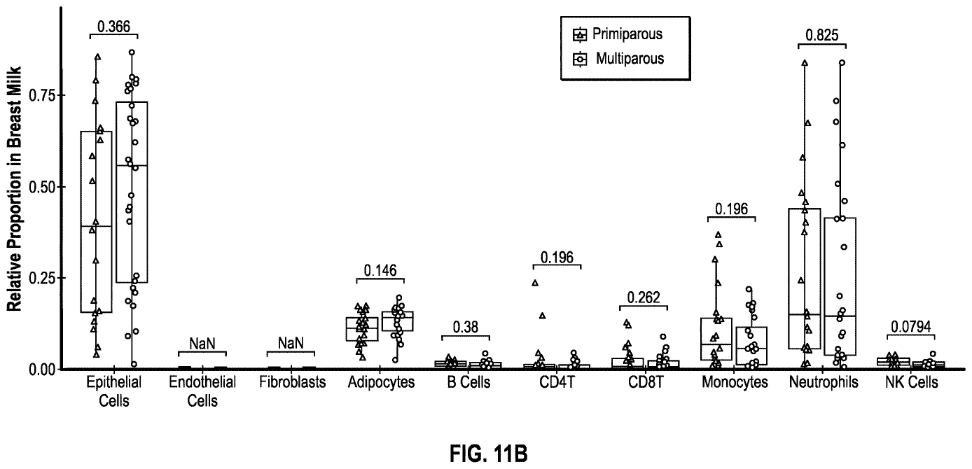

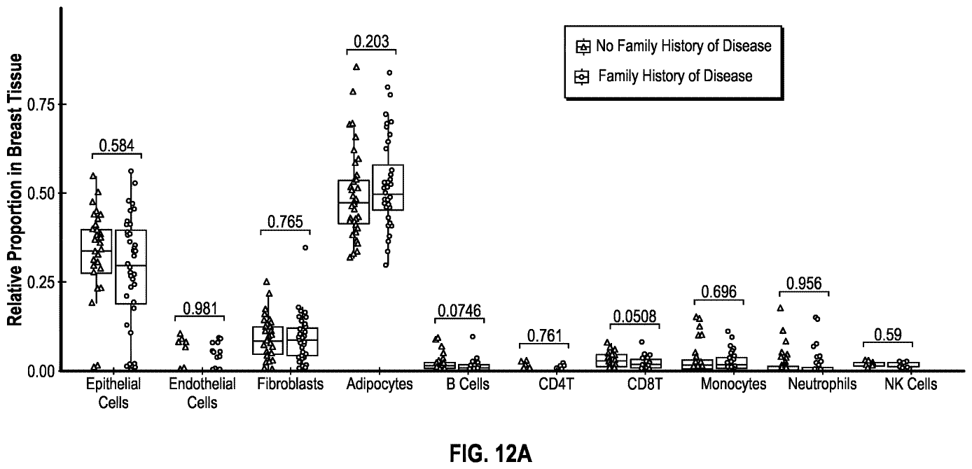

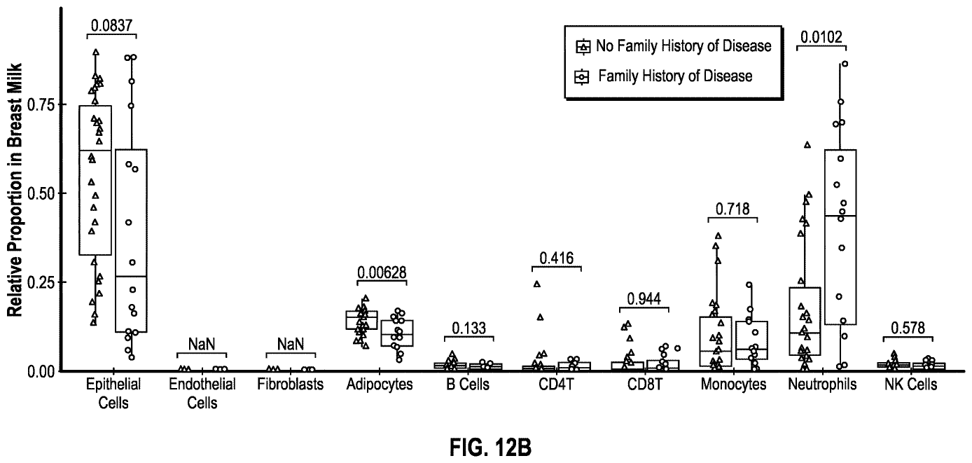

Parity is significantly negatively associated with B cell proportions (P<0.05), and significantly positively associated with fibroblast and NK cell proportions (P<0.05, FIGS. 11A and 11B). Family history of disease was not significantly associated with the relative proportion of cell types (FIGS. 12A and 12B).

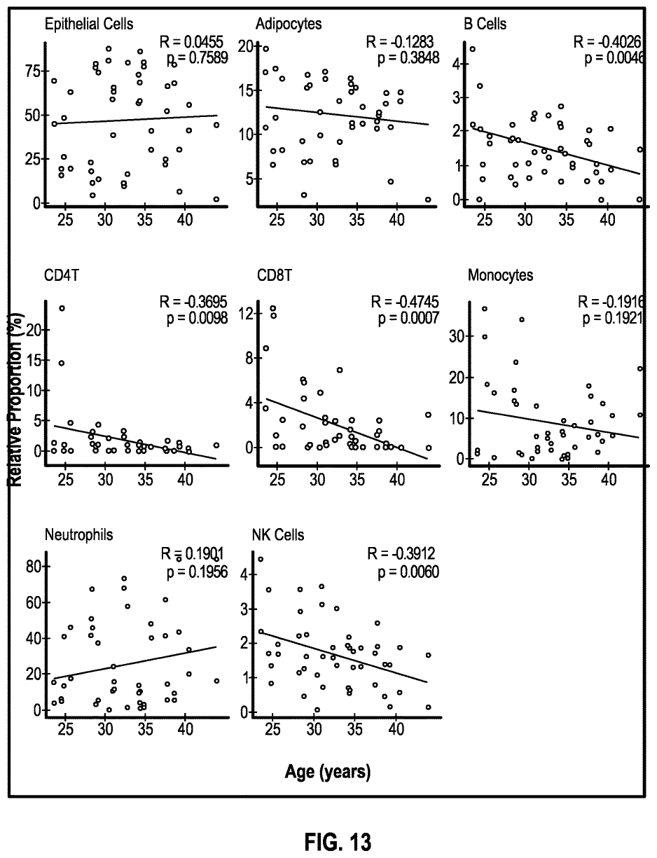

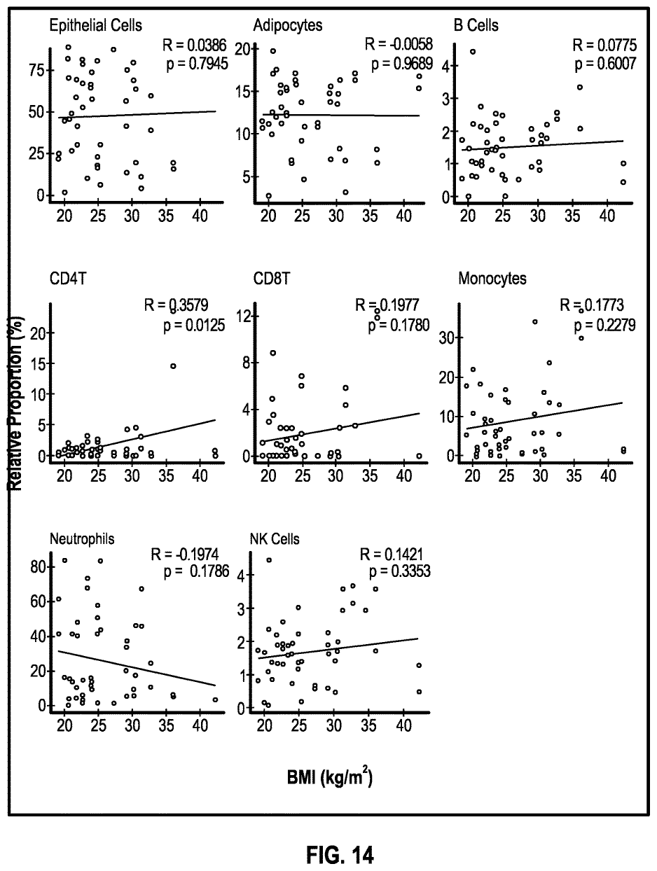

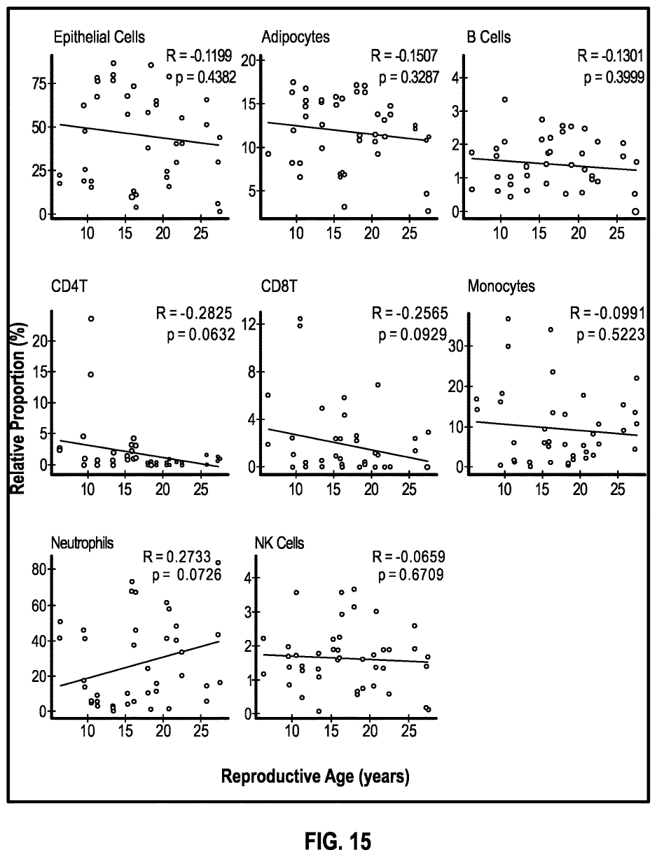

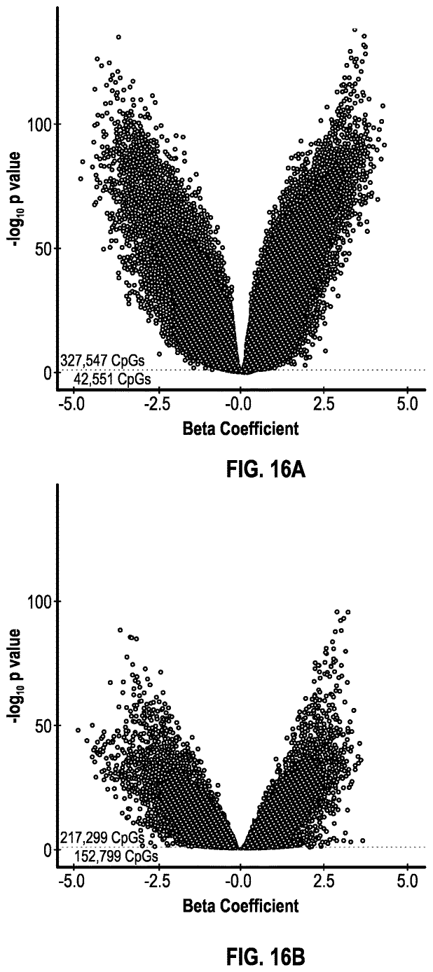

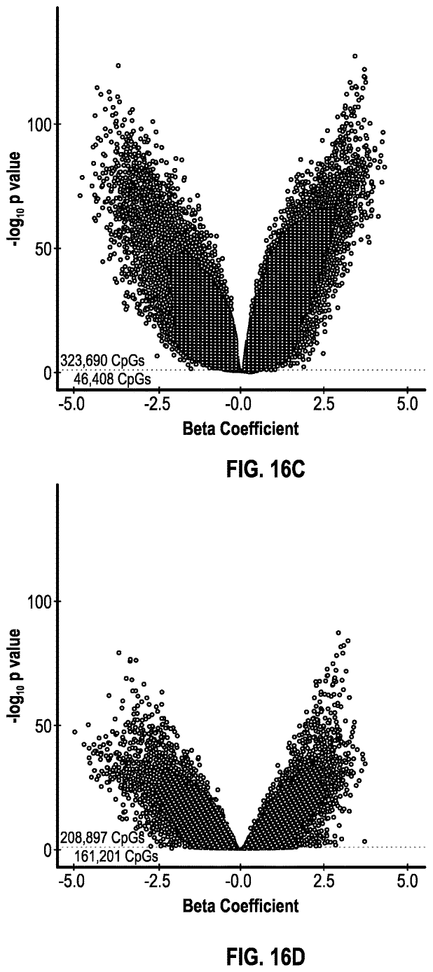

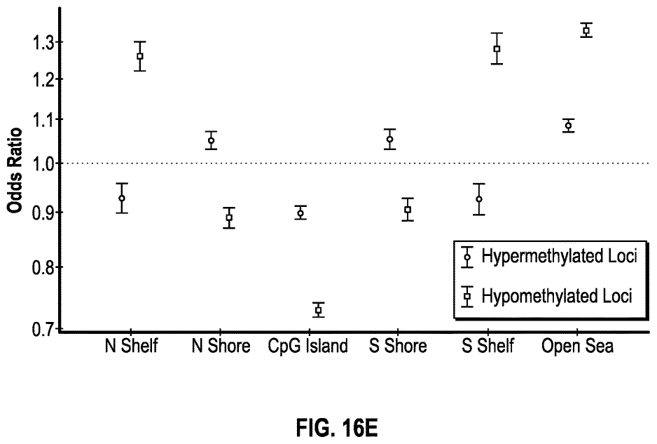

In breast milk, cellular composition is associated with breast cancer risk factors. Age was significantly negatively associated with the relative proportion of B cells, CD4+ T cells, CD8+ T cells, and NK cells (P<0.05, FIG. 13). BMI was significantly positively associated CD4+ T cell proportions (P<0.05, FIG. 14). Family history of disease was significantly negatively associated with adipocyte proportions (P<0.05) and significantly positively associated with neutrophil proportions (P<0.05. FIGS. 12A and 12B). No significant associations were observed between cell-type proportions and reproductive age (FIG. 15) or parity (FIGS. 16A-16B).

### DNA Methylation Differences Between Solid Breast Tissue and Breast Milk are Largely Attributable to Differences in Cellular Composition.

Comparing the methylome of breast milk to that of solid breast tissue, 327,547 CpG loci were identified as differentially methylated (FDR<0.05) after adjusting for subject age and parity (FIG. 16A). After adjusting for cellular composition, specifically the relative proportions of epithelial cells, endothelial cells, fibroblasts, adipocytes, monocytes and neutrophils in each sample, observed differential methylation was strongly attenuated (FIG. 16B). These findings were robust to sensitivity analyses also adjusting for family history of disease (FIGS. 16C-16D) to account for underlying differences in the proportion of subjects with a family history of disease between the two cohorts. In age, parity, and cell type adjusted models, hypermethylated CpG loci in breast milk relative to solid breast tissue were significantly enriched for CpG island bordering shore regions and CpG sparse open sea regions, while depleted for CpG dense CpG island regions. Similarly, hypomethylated loci were enriched for open sea regions and depleted for CpG islands but also depleted for shore regions (FIG. 16C).

Shared Associations of DNA Methylation with Reproductive Age, Family History of Breast Cancer, and BMI in Solid Breast Tissue and Breast Milk.

Reproductive age is used to assess a possible association between reproductive history, including age at menarche and age at first birth, and breast DNA methylation. Reproductive age was defined as the difference between age at first birth and age at menarche for parous women and between age at donation and age at menarche for nulliparous women.

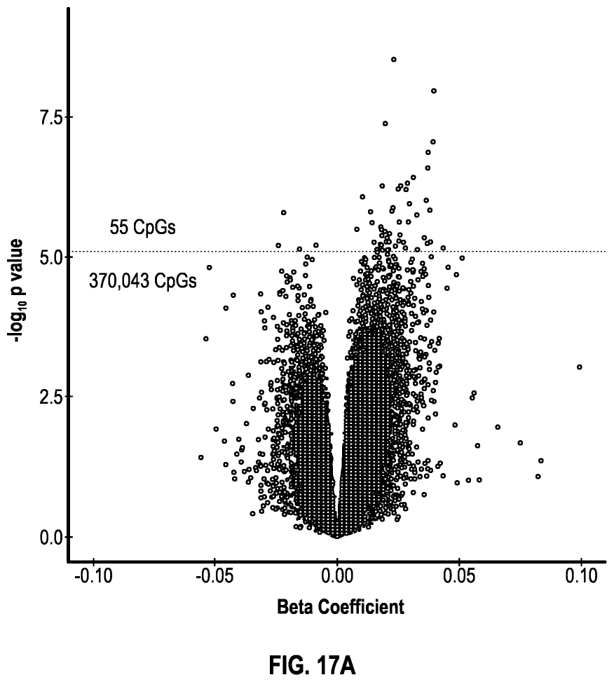

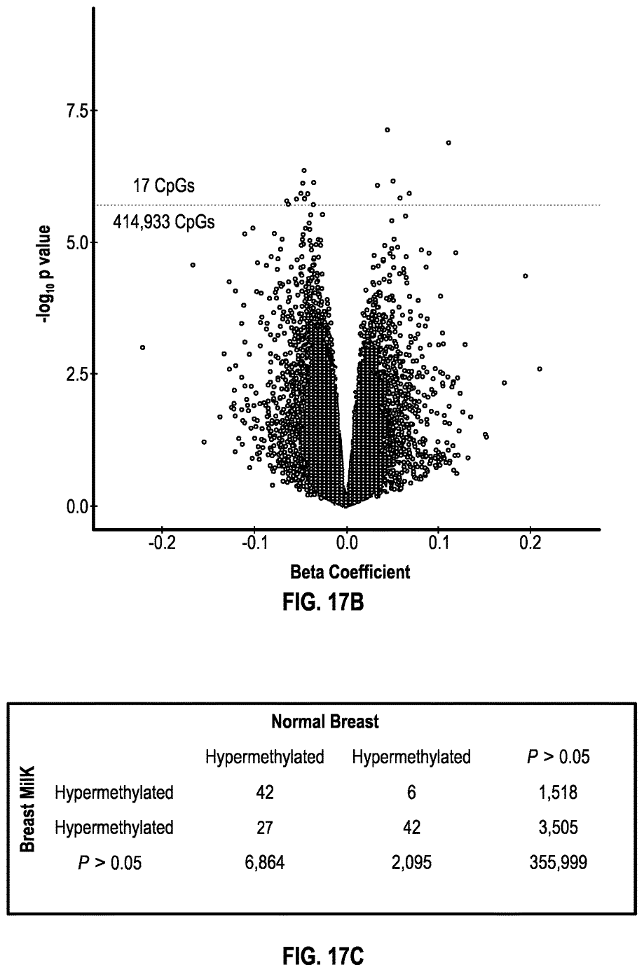

Reproductive age was significantly associated (FDR<0.05) with methylation status of 55 CpG loci in solid breast tissue (FIG. 17A) and 17 CpG loci in breast milk (FIG. 17B), after adjusting for differences in underlying cellular composition. At a nominal significance threshold of P<0.01, 9,076 loci were identified as differentially methylated with increasing reproductive age in solid breast tissue and 5,140 loci in breast milk. In both solid breast tissue and breast milk. 42 hypermethylated and 42 hypomethylated CpG loci had shared differential methylation in the two tissue types (FIG. 17C). Of the shared hypermethylated loci, 4 tracked to a region within GRM2 while 2 of the shared hypomethylated loci tracked to RASA3, a member of the Ras pathway. These findings were robust to sensitivity analyses adjusting for parity in solid tissue samples due to the presence of nulliparous women in that cohort and the differences in calculation of reproductive age relative to parity.

Approximately half of the solid breast tissue samples and a third of the breast milk samples were collected from women with a family history of breast or ovarian cancer (Table 1).

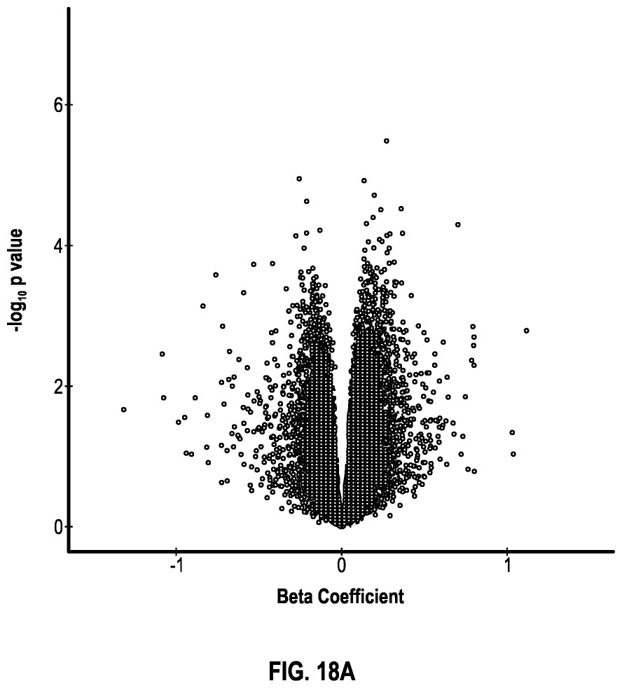

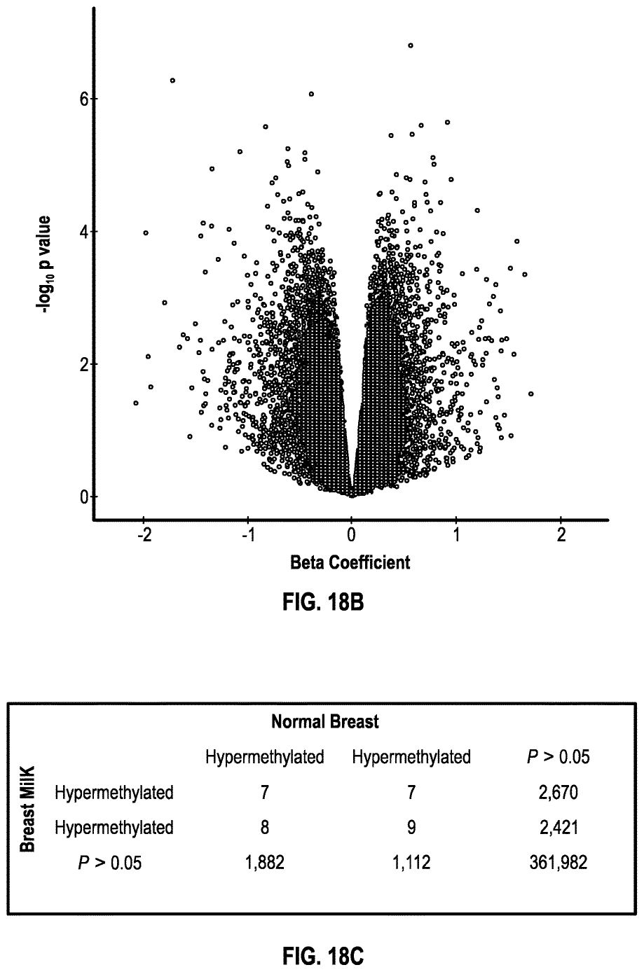

When assessing the association between family history of breast or ovarian cancer (binary) and DNA methylation, no CpG loci met a significance threshold of FDR<0.05 in either solid breast tissue or breast milk. At a nominal significance threshold of P<0.01, 3,025 loci were identified as differentially methylated in solid breast tissue (FIG. 18A) and 25,434 loci were identified as differentially methylated in breast milk (FIG. 18B). Overlap was observed in the identified differentially methylated loci with 7 loci hypermethylated in both solid breast tissue and breast milk and 9 loci hypomethylated, including a locus mapping to the gene body region of BRCA2, in both tissues (FIG. 18C).

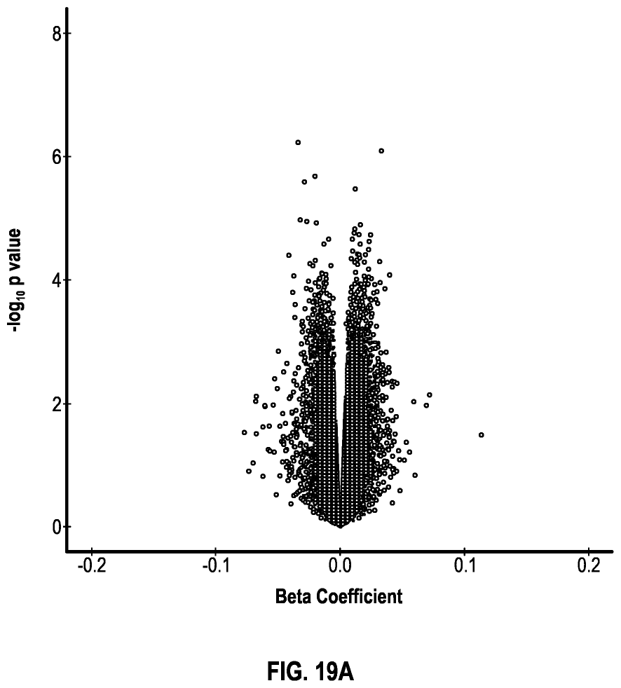

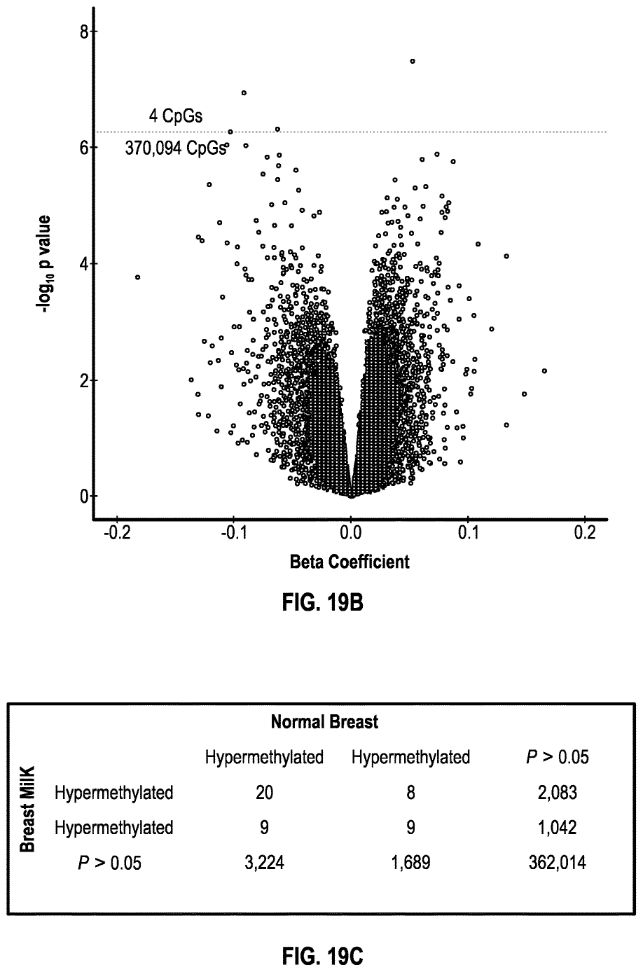

When assessing the association between pre-pregnancy BMI and breast milk DNA methylation, 4 CpG loci were identified as differentially methylated at a significance threshold of FDR<0.05 (FIG. 19A). Among these 4 loci, 3 demonstrated decreased gene body methylation in HLA-DQA2, VAC14, and TBC1D22A, respectively, with increasing pre-pregnancy BMI. While no CpG loci met a significance threshold of FDR<0.05 when assessing the association between BMI and solid breast tissue DNA methylation (FIG. 19B), 20 CpG loci were consistently hypermethylated with increasing BMI across tissue types and 9 CpG loci were consistently hypomethylated (FIG. 19C).

### Hypermethylated CpG Loci in Solid Breast Tissue and Breast Milk of Parous Women are Enriched for CpG Sparse Open Sea Regions.

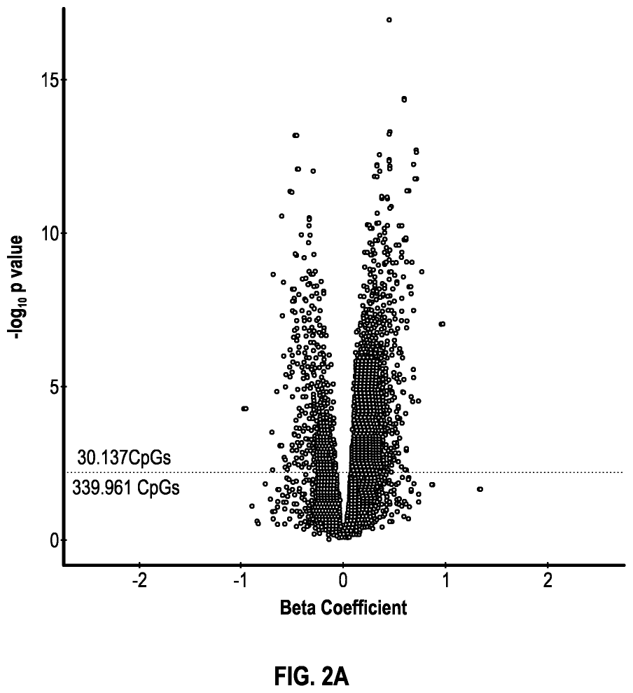

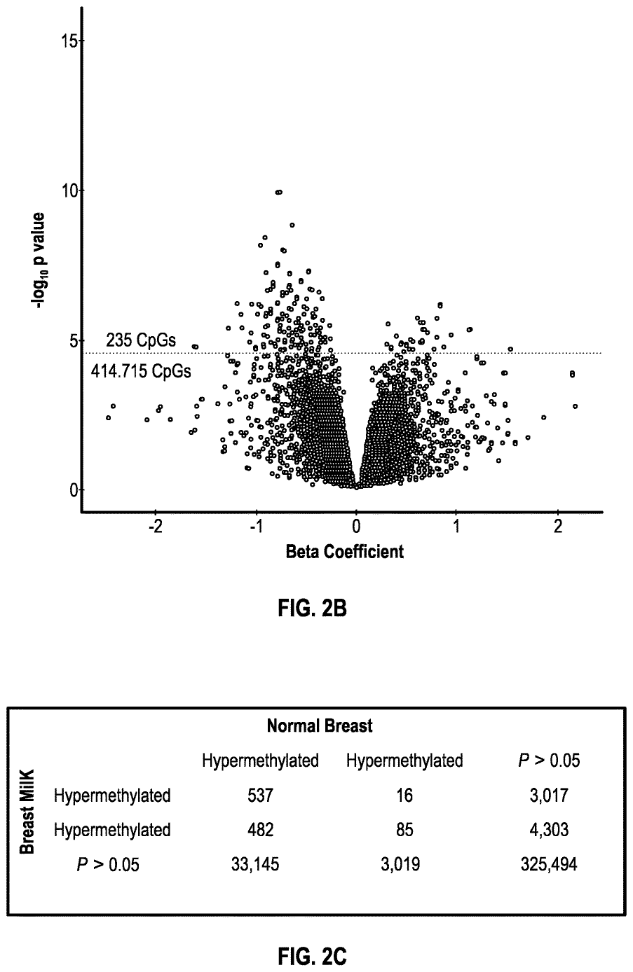

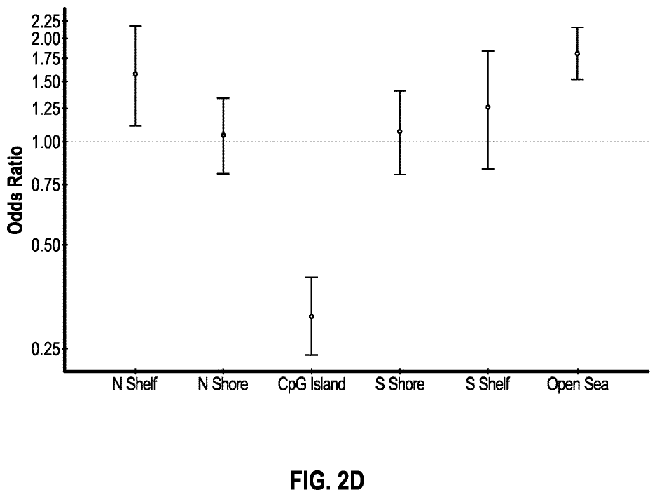

In solid breast tissue, 30,137 loci are identified as differentially methylated (FDR<0.05) in women with a history of at least one live birth relative to nulliparous women after adjusting for the estimated cellular composition of each sample (FIG. 2A). In breast milk, 235 CpG loci were identified as differentially methylated (FDR<0.05) in women with at least one prior live birth relative to women for which the current pregnancy was their first live birth (FIG. 2B). At a nominal significance threshold of P<0.01, 37,284 loci were identified as differentially methylated in solid breast tissue and 8.440 loci were identified as differentially methylated in breast milk with 537 loci being consistently hypermethylated between the two tissue types (FIG. 2C). These 537 hypermethylated loci demonstrated 1.8-fold enrichment for CpG sparse open sea regions (95% CI: 1.5, 2.2) and 0.3-fold depletion for CpG dense CpG island regions (95% CI: 0.2, 0.4; FIG. 2D). The 85 hypomethylated loci included 5 loci mapping to the promoter region of tumor suppressor gene TAGLN.

CpG Loci that are Hypermethylated in Solid Breast Tissue and Breast Milk with Increasing Age are Enriched for CpG Island Bordering Shore Regions.

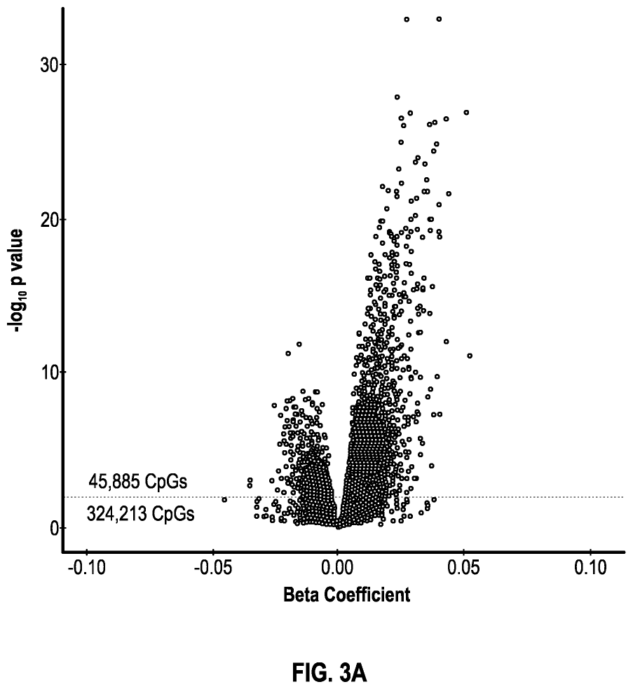

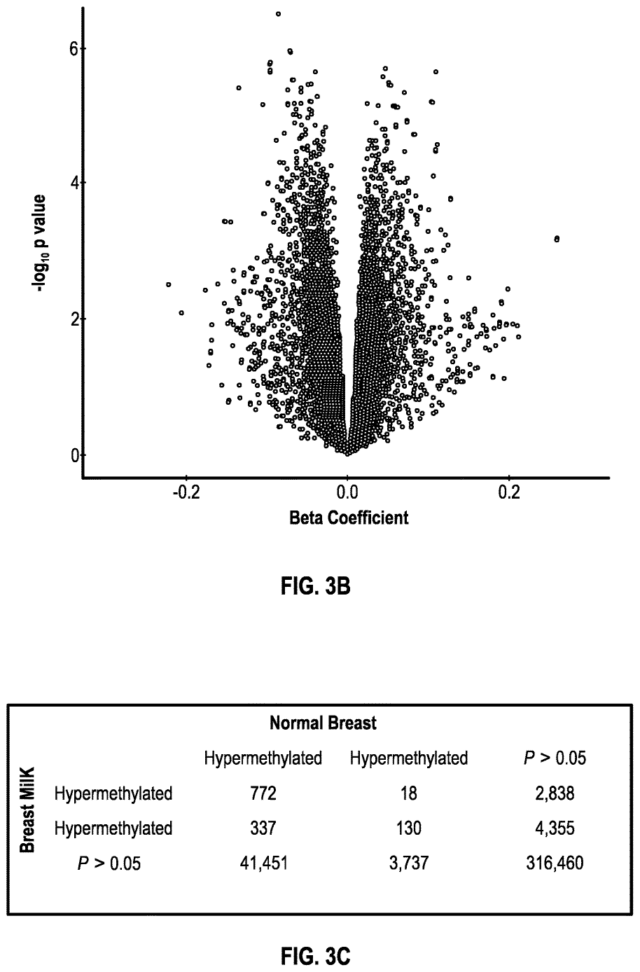

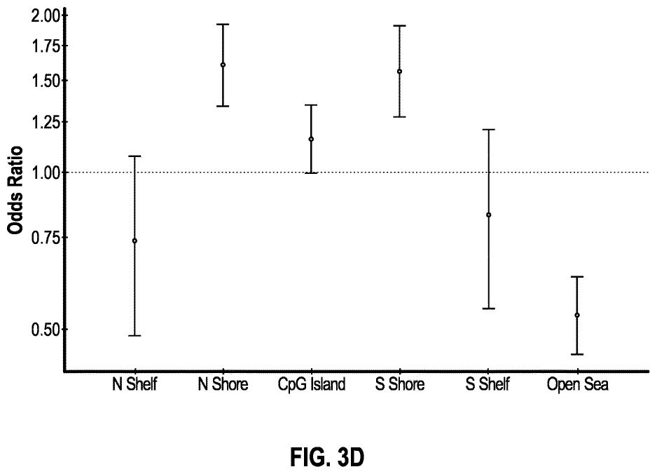

In solid breast tissue, the methylation levels at 45,885 loci was significantly associated with age at donation after adjusting for the estimated cellular composition of each sample (Q<0.05; FIG. 3A). In breast milk, no CpG loci demonstrated an association between methylation status and age at an FDR significance threshold of (Q<0.05; FIG. 3B). Due to the markedly smaller sample size and the expected narrower age range of subjects who donated breast milk (Table 1), a nominal significance threshold of P<0.01 was used for subsequent comparisons. At a nominal significance threshold of P<0.01 and adjusting for sample cellular composition, 46,445 loci were associated with age in solid breast tissue and 8,450 loci were associated with age in breast milk. Among these differentially methylated loci, 130 loci were consistently hypomethylated with increasing age in both tissue types and 772 loci were consistently hypermethylated with increasing age (FIG. 3C). The 772 consistently hypermethylated loci were significantly enriched for CpG island bordering shore regions (OR=1.6; 95% CI: 1.3, 1.9; FIG. 3D). Due to age and parity being positively correlated, associations were additionally assessed in models adjusting for both chronological age and parity. In the combined model, fewer loci were identified as being consistently hyper- or hypomethylated in both tissue types associated with age and parity, respectively.

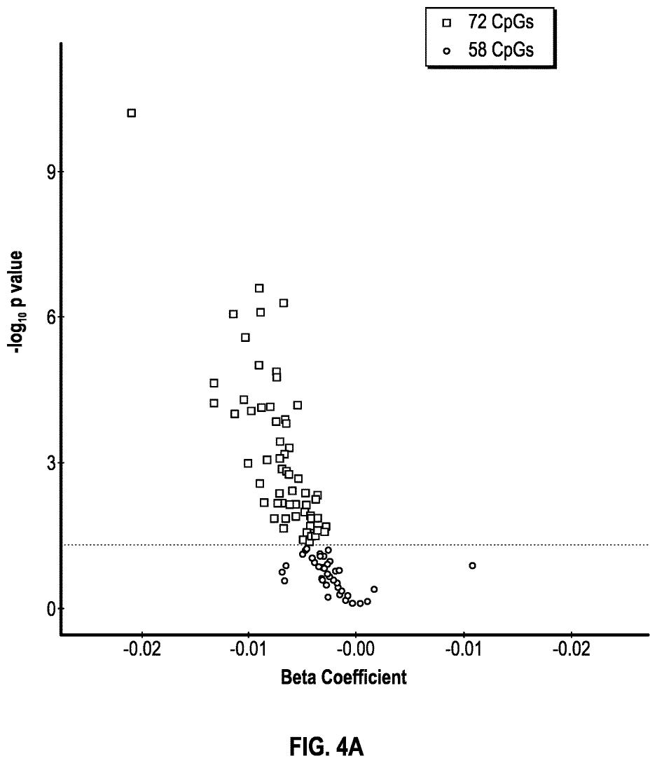

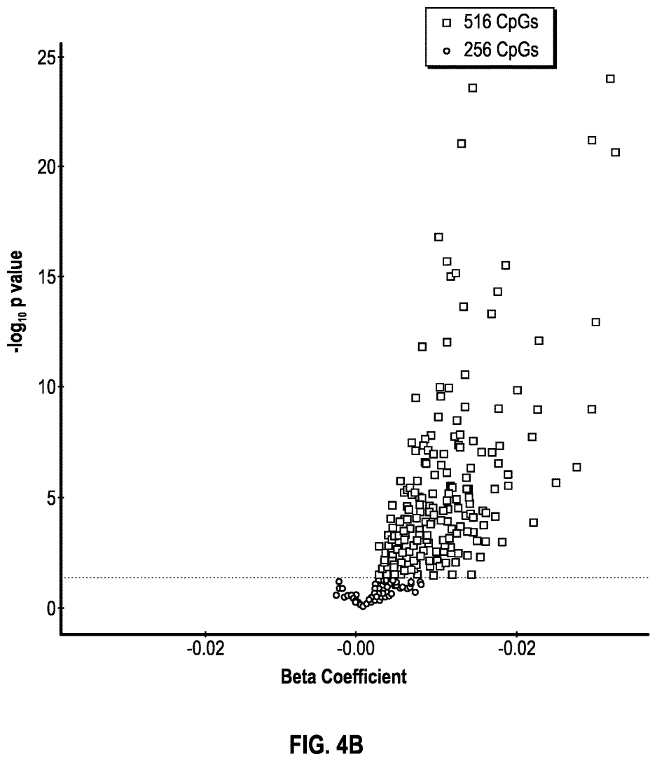

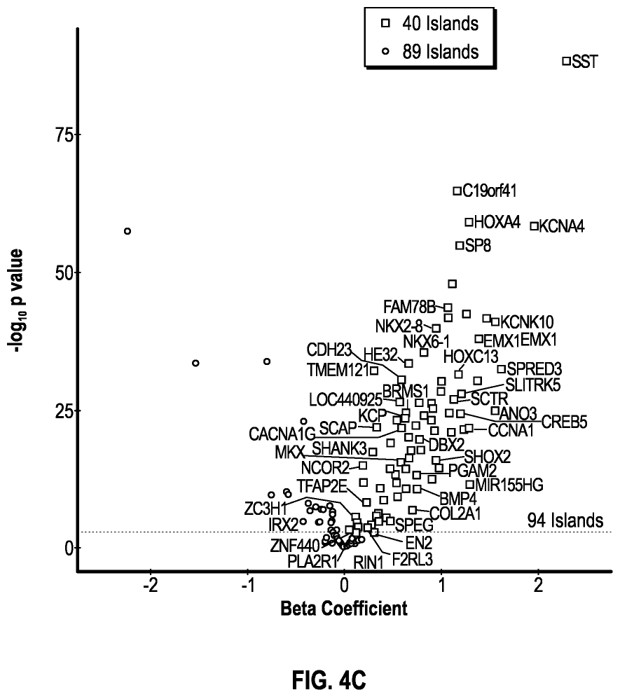

To replicate age-related findings with cell-type adjustment, publicly available data (GSE101961) from a study assessing the association between age and DNA methylation (measured on the Illumina 450 k array) in breast tissue from 121 disease-free donors (27) is accessed. A total of 588 CpG age-related loci replicated in the independent data set, including 72 hypomethylated loci (55%) and 516 hypermethylated loci (67%; FIGS. 4A-4B). Further, the validation results are compared using a subset of 130 and 772 randomly selected CpG loci. Only 21 hypomethylated loci (16%), and 113 hypermethylated loci (15%) are identified as related with age.

CpG Islands with Shores that Contain Age-Associated Hypermethylated Loci Demonstrate Hypermethylation in Tumor Relative to Adjacent Normal Tissue.

From the age-associated methylation results, there were 223 CpG island shores that had significant age-associated DNA methylation in both breast tissue and breast milk samples. The study then tests whether the neighboring CpG island exhibited hypermethylation in breast tumors using 450 k array data from TCGA tumors (n=392) and adjacent normal tissue (n=82). 94 CpG islands (42% of the 223 assessed island regions) are identified with significant hypermethylation in breast tumors compared with adjacent normal tissue (FIG. 4C).

Among these CpG islands, the most hypermethylated island in tumor relative to adjacent normal tissue mapped to the promoter region of SST.

### SFRP2 Age-Associated Promoter Hypermethylation is Observed in Tumor and Adjacent Normal Tissue.

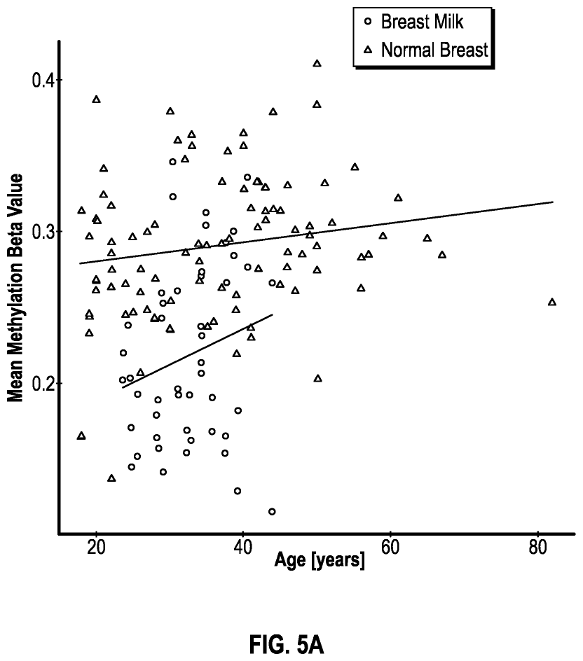

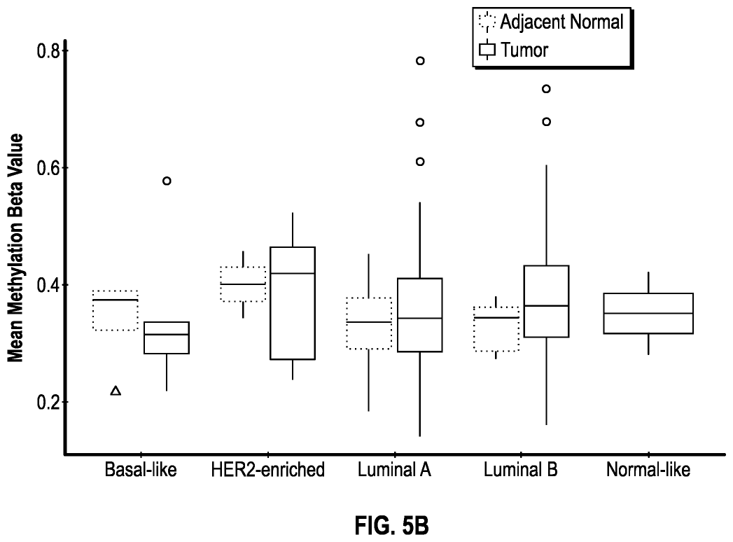

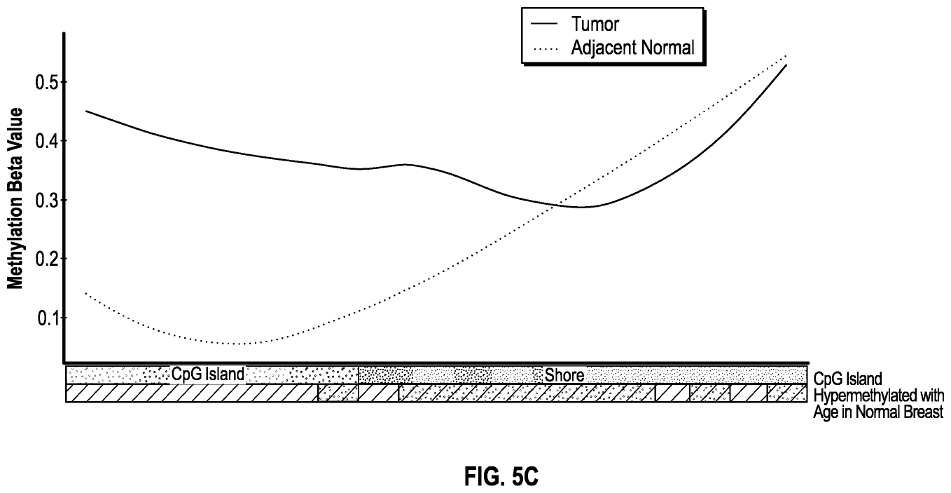

Of the 772 CpG loci hypermethylated in both solid breast tissue and breast milk. 10 loci mapped to the promoter region of SFRP2, the most overlapping loci to map to a single gene. The next most hypermethylated genes were GRM2 with 7 overlapping hypermethylated loci in the promoter CpG island and HOXC13 with 5 overlapping hypermethylated loci in the gene body. Of the 10 hypermethylated loci in SFRP2, 9 mapped to the CpG island shore region within the promoter. The mean methylation beta value of the 10 hypermethylated loci in each sample positively correlates with age across both solid breast tissue and breast milk (FIG. 5A). Mean methylation of these 10 loci also demonstrate intermediate methylation levels in both tumor and adjacent normal tissue, independent of breast cancer subtype, in TCGA data (FIG. 5B). Importantly, across the promoter region of SFRP2 in breast tumor and adjacent normal tissue from TCGA, intermediate methylation levels are observed in the CpG island shore. In contrast, intermediate methylation of the CpG island itself is only observed in tumor tissue (FIG. 5C). Thus, methylation profiles of promoter CpG island shore SFRP2 in breast tumor and adjacent normal tissue are breast cancer subtype-independent.

### DISCUSSION

Using genome-scale DNA methylation data, the associations of established breast cancer risk factors are compared with DNA methylation in both solid breast tissue and breast milk. Using a novel reference library for cell-type-specific DNA methylation in breast tissue, differences in the cellular composition of solid breast tissue and breast milk are identified. As references for breast epithelial cells, endothelial cells, and fibroblasts were derived from cell lines, there remains the possibility that their methylome differs from that of cells isolated from tissue. While this approach remains advantageous over reference-free approaches, additional work is needed to further develop breast specific reference libraries for component cell types.

Solid tissue was found to have a higher relative proportion of adipocytes while breast milk had higher proportions of epithelial cells and immune cells. This reference library was critical in adjusting for cellular composition in downstream analyses to allow for direct comparisons between breast milk and solid breast tissue without the analyses being confounded by differences in the underlying cellular composition of each tissue type.

Additionally, identified statistically significant associations between estimated sample cellular composition and investigated breast cancer risk factors in both solid breast tissue and breast milk reinforces the importance of adjusting for sample cellular composition in downstream analyses due to the risk of confounding of results by differences in cellular composition in the absence of adjustment.

While the scope of shared differentially methylated loci associated with reproductive age, family history of disease, and BMI observed in both tissue types was narrow, small sample size, particularly in the breast milk data, may have precluded the identification of differentially methylated loci. Furthermore, despite little observed overlap, family history of disease was associated with gene body hypomethylation, typically associated with reduced gene expression, of well documented tumor suppressor gene BRCA2 across tissue types. This suggests that relevant molecular alterations associated with disease risk in breast tissue may be detectable in breast milk, a far less invasive biospecimen for stratifying disease risk. Furthermore, while the use of breast milk as a potential biomarker of disease risk is limited to lactating women, findings in breast milk have the potential to be extended to nipple aspirate fluid collected from non-lactating women (28).

Despite minimal overlap observed in differentially methylated CpG loci with increasing BMI in breast milk and solid breast tissue, breast milk samples interesting identified 4 differentially methylated CpG loci with increasing BMI after correcting for multiple comparisons while no differentially methylated CpG loci were observed in solid breast tissue at this significance threshold. One identified CpG locus mapped to the gene body region of TBC1D22A, a gene previously linked to obesity (29). This highlights the potential to identify risk-factor associated molecular alterations in breast milk and motivates further investigation of this potential biospecimen in identifying alterations indicative of disease risk.

More extensive shared differentially methylated loci with increasing age and with parity were observed between solid breast tissue and breast milk. Shared hypermethylated loci associated with parity were enriched for CpG island sparse open sea regions which commonly overlap with enhancer regions, suggesting that these alterations to DNA methylation associated with parity, which is associated with a decreased risk of breast cancer, may play an important role in gene regulation.

Shared hypermethylated loci with increasing age in solid breast tissue and breast milk are significantly enriched for CpG island shore regions. Previous work has identified that CpG island hypermethylation observed in tumor tissue relative to adjacent normal tissue may begin in CpG island bordering shore regions (14, 30). Alterations in shore regions would be undetected in comparisons of tumor relative to adjacent normal tissue as they are also present in the adjacent normal tissue itself. Therefore, the identification of CpG island shore hypermethylation in non-diseased tissue with increasing age may be indicative of early alterations in DNA methylation associated with increased risk of disease.

To further investigate this, the methylation status of CpG islands in tumor relative to adjacent normal TCGA tissue is assessed for islands with shore loci that were hypermethylated with age to explore the possibility of seeding hypermethylation events in CpG island shores in normal tissue leading to encroachment of hypermethylated CpG islands in tumor tissue. CpG island hypermethylation was observed for CpG island shores that were hypermethylated with increasing age in solid breast tissue and breast milk.

Notably, the most hypermethylated of the assessed CpG islands mapped to the promoter region of SST which encodes the hormone somatostatin (SST), a hormone that is elevated during pregnancy and lactation (31) and that is involved in the indirect inhibition of mammary tumor growth through the inhibition of hormones and growth factors that promote tumor growth (32). Additionally, the promoter CpG island of SST was found to exhibit hypermethylation in the CpG island shore in adjacent normal tissue relative to paired tissue collected from the opposite breast and subsequent hypermethylation of the CpG island itself in paired tumor tissue (14).

SFRP2 is identified as the gene with the greatest number of shared hypermethylated loci with increasing age in solid breast tissue and breast milk. SFRP2 encodes secreted frizzled-related protein 2, an antagonist of the Wnt pathway, the secretion of which has been documented as being decreased in multiple tumor types including breast tumors (33).

Furthermore, promoter hypermethylation of SFRP2 has been identified as a mechanism of decreased protein expression in breast tumor tissue and has even been proposed as a potential tumor biomarker (34).

Of the 10 shared hypermethylated loci identified in SFRP2, 9 mapped to the CpG island shore in the promoter region while the remaining locus mapped to the adjacent promoter CpG island. In TCGA breast tumor and adjacent normal methylation data, similar intermediate levels of methylation at shore loci in both tumor tissue and adjacent normal tissue are identified, while increased methylation of the adjacent island was observed only in tumor tissue, with these loci remaining hypomethylated in adjacent normal tissue. Taken together, these findings may suggest that DNA methylation alterations to the promoter CpG island shore region of SFRP2 occur early in carcinogenesis, priming the adjacent CpG island for subsequent hypermethylation. Early alterations to shore region sites have the potential to act as biomarkers for disease risk.

The study was limited by the small sample size, particularly in the breast milk data, as well as the unpaired nature of solid breast tissue and breast milk samples, due to the difficulty and ethical concerns of collecting breast biopsies from healthy lactating mothers, preventing direct comparisons between tissue types. However, developing a breast-tissue-specific reference library for the estimation of cellular composition greatly enhanced the ability to compare results across tissue types with minimal confounding by differences in underlying cellularity. Despite these limitations, the system and method herein successfully identifies common breast cancer risk factor-associated DNA methylation alterations in both solid breast tissue and breast milk.

### Computing Environment

Referring to FIG. 20, it should be clear that the library and patient-specific information/data herein 2010 can be stored and accessed via a computer process(or) environment 1740 that includes appropriate user interfaces (display 1750, mouse 1749, and keyboard 1747) instantiated on local/remote computing devices 1748, such as PCs laptops, servers, tablets, smartphones, etc. The remote computing devices can be connected via an appropriate network 1752, such as the well-known Internet. In a generalized processing architecture, as shown, the data of libraries and users is handled via a database process(or) 1742. The data can be stored in distributed, networked environments, such as a cloud computing arrangement. Users of access and manipulate data, and receive results, based upon an interface process(or) 1744 the can be implement according to conventional or custom arrangements. The library and associated data can be made accessible to users based upon an open source model, or a subscription model, in which the users provide credentials based upon a currently available/custom access control arrangement (e.g. a paid-up subscription or granted fee access). Appropriate security protocols can also be employed (SSL, encryption, etc.) to maintain secrecy as to users and/or transmitted data. More particularly, libraries can be tailored in appropriate databases to cross-referenced patient conditions so that relevant results on the particular condition are accessed. The handling of data can be implemented using a variety of procedures embodied herein by a results process(or) 1746. Mechanisms for appending data to a library and/or associating the data with a particular condition should be clear to those of skill in the art.

### REFERENCES AS DENOTED IN BRACKETED REFERENCE NUMERALS ABOVE

1. Siegel R L, Miller K D, Fuchs H E, Jemal A. Cancer Statistics, 2021. CA: A Cancer Journal for Clinicians. 2021; 71.
2. Dumitrescu R G, Cotarla I. Understanding breast cancer risk—Where do we stand in 2005? Journal of Cellular and Molecular Medicine. 2005.
3. Barnard M E, Boeke C E, Tamimi R M. Established breast cancer risk factors and risk of intrinsic tumor subtypes. Biochimica et Biophysica Acta—Reviews on Cancer. Elsevier B.V.; 2015. page 73-85.
4. Maas P, Barrdahl M, Joshi A D, Auer P L, Gaudet M M, Milne R L, et al. Breast Cancer Risk From Modifiable and Nonmodifiable Risk Factors Among White Women in the United States. JAMA Oncol. 2016; 2.
5. Michailidou K, Hall P, Gonzalez-Neira A, Ghoussaini M, Dennis J, Milne R L, et al. Large-scale genotyping identifies 41 new loci associated with breast cancer risk. Nature Genetics. 2013; 45.
6. Michailidou K, Beesley J, Lindstrom S, Canisius S. Dennis J, Lush M J, et al. Genome-wide association analysis of more than 120,000 individuals identifies 15 new susceptibility loci for breast cancer. Nature Genetics. 2015; 47.
7. Michailidou K, Lindstrom S, Dennis J, Beesley J, Hui S, Kar S, et al. Association analysis identifies 65 new breast cancer risk loci. Nature. 2017; 551.
8. Lee A, Mavaddat N, Wilcox A N, Cunningham A P, Carver T, Hartley S, et al. BOADICEA: a comprehensive breast cancer risk prediction model incorporating genetic and nongenetic risk factors. Genetics in Medicine. 2019:21.
9. Kelsey J L, Gammon M D, John E M. Reproductive factors and breast cancer. Epidemiologic Reviews. 1993; 15.
10. Phipps A I, Buist D S M, Malone K E, Barlow W E, Porter P L, Kerlikowske K, et al. Reproductive history and risk of three breast cancer subtypes defined by three biomarkers. Cancer Causes and Control. 2011; 22.
11. Koboldt D C, Fulton R S, McLellan M D, Schmidt H, Kalicki-Veizer J, McMichael J F, et al. Comprehensive molecular portraits of human breast tumours. Nature [Internet]. Nature Publishing Group; 2012 [cited 2019 Mar. 4]; 490:61-70. Available from: http://www.nature.com/doifinder/10.1038/nature11412.
12. Johnson K C, Koestler D C, Fleischer T, Chen P, Jenson E G, Marotti J D, et al. DNA methylation in ductal carcinoma in situ related with future development of invasive breast cancer. Clinical Epigenetics [Internet]. BioMed Central; 2015 [cited 2019 Mar. 5]; 7:75. Available from: http://www.clinicalepigeneticsjoumal.com/content/7/1/75.
13. Fleischer T, Frigessi A, Johnson K C, Edvardsen H, Touleimat N, Klajic J, et al. Genome-wide DNA methylation profiles in progression to in situ and invasive carcinoma of the breast with impact on gene transcription and prognosis. Genome Biology [Internet]. BioMed Central; 2014 [cited 2019 May 6]; 15:435. Available from: http://genomebiology.biomedcentral.com/articles/10.1186/s13059-014-0435-x
14. Muse M E, Titus A J, Salas L A, Wilkins O M, Mullen C, Gregory K J, et al. Enrichment of CpG island shore region hypermethylation in epigenetic breast field cancerization. Epigenetics. 2020; 15.
15. Johnson K C, Houseman E A, King J E, Christensen B C. Normal breast tissue DNA methylation differences at regulatory elements are associated with the cancer risk factor age. Breast Cancer Res [Internet]. BioMed Central; 2017 [cited 2017 Aug. 2]; 19:81. Available from: http://www.ncbi.nlm.nih.gov/pubmed/28693600.
16. Salas L A, Lundgren S N, Browne E P, Punska E C, Anderton D L, Karagas M R, et al. Prediagnostic breast milk DNA methylation alterations in women who develop breast cancer. Human Molecular Genetics. 2020; 29.
17. Gilbert-Diamond D, Cottingham K L, Gruber J F, Punshon T, Sayarath V, Gandolfi A J, et al. Rice consumption contributes to arsenic exposure in U S women. Proc Natl Acad Sci USA [Internet]. National Academy of Sciences; 2011 [cited 2017 Aug. 20]; 108:20656-60. Available from: http://www.ncbi.nlm.nih.gov/pubmed/22143778.
18. Aryee M J, Jaffe A E, Corrada-Bravo H, Ladd-Acosta C, Feinberg A P, Hansen K D, et al. Minfi: a flexible and comprehensive Bioconductor package for the analysis of Infinium DNA methylation microarrays. Bioinformatics [Internet]. Springer, New York; 2014 [cited 2017 Jun. 29]:30:1363-9. Available from: https://academic.oup.com/bioinformatics/article-lookup/doi/10.1093/bioinformatics/btu049.
19. Teschendorff A E, Marabita F, Lechner M, Bartlett T, Tegner J, Gomez-Cabrero D, et al. A beta-mixture quantile normalization method for correcting probe design bias in Illumina Infinium 450 k DNA methylation data. Bioinformatics [Internet]. 2013 [cited 2018 Apr. 10]; 29:189-96. Available from: https://academic.oup.com/bioinformatics/article-lookup/doi/10.1093/bioinformatics/bts680.
20. Zhou W, Laird P W, Shen H. Comprehensive characterization, annotation and innovative use of Infinium DNA methylation BeadChip probes. Nucleic Acids Res. 2017; 45.
21. Holm K, Staaf J, Lauss M, Aine M, Lindgren D, Bendahl P-O, et al. An integrated genomics analysis of epigenetic subtypes in human breast tumors links DNA methylation patterns to chromatin states in normal mammary cells. Breast Cancer Research [Internet]. BioMed Central; 2016 [cited 2019 May 31]; 18:27. Available from: http://breast-cancer-research.biomedcentral.com/articles/10.1186/s13058-016-0685-5.
22. Amer P, Sinha I, Thorell A, Rydén M, Dahlman-Wright K, Dahlman I. The epigenetic signature of subcutaneous fat cells is linked to altered expression of genes implicated in lipid metabolism in obese women. Clinical Epigenetics. 2015:7.
23. Salas L A, Koestler D C, Butler R A, Hansen H M, Wiencke J K, Kelsey K T, et al. An optimized library for reference-based deconvolution of whole-blood biospecimens assayed using the Illumina HumanMethylationEPIC BeadArray. Genome Biology [Internet]. BioMed Central; 2018 [cited 2018 Jun. 24]; 19:64. Available from: https://genomebiology.biomedcentral.com/articles/10.1186/s13059-018-1448-7
24. Koestler D C, Jones M J, Usset J, Christensen B C, Butler R A, Kobor M S, et al. Improving cell mixture deconvolution by identifying optimal DNA methylation libraries (IDOL). BMC Bioinformatics [Internet]. BioMed Central; 2016 [cited 2018 Jun. 26]; 17:120. Available from: http://www.biomedcentral.com/1471-2105/17/120.
25. Teschendorff A E, Relton C L. Statistical and integrative system-level analysis of DNA methylation data. Nature Reviews Genetics [Internet]. Nature Publishing Group; 2017 [cited 2018 Nov. 3]; 19:129-47. Available from: http://www.nature.com/doifinder/10.1038/nrg.2017.86.
26. Newman A M, Liu C L, Green M R, Gentles A J, Feng W, Xu Y, et al. Robust enumeration of cell subsets from tissue expression profiles. Nature Methods. 2015; 12.
27. Song M A. Brasky T M, Weng D Y, McElroy J P, Marian C. Higgins M J, et al. Landscape of genome-wide age-related DNA methylation in breast tissue. Oncotarget. 2017; 8.
28. Wrensch M R, Petrakis N L, Gruenke L D, Emster V L, Miike R. King E B, et al. Factors associated with obtaining nipple aspirate fluid: Analysis of 1428 women and literature review. Breast Cancer Research and Treatment. 1990; 15.
29. Liu A Y, Gu D, Hixson J E, Rao D C, Shimmin L C, Jaquish C E, et al. Genome-wide linkage and regional association study of obesity-related phenotypes: The GenSalt study. Obesity. 2014; 22.
30. Skvortsova K, Masle-Farquhar E, Luu P-L, Song J Z, Qu W, Zotenko E. et al. DNA Hypermethylation Encroachment at CpG Island Borders in Cancer Is Predisposed by H3K4 Monomethylation Patterns. Cancer Cell [Internet]. Elsevier; 2019 [cited 2019 Apr. 10]; 35:297-314.e8. Available from: http://www.ncbi.nlm.nih.gov/pubmed/30753827
31. Goldstein A, Armony-Sivan R, Rozin A, Weller A. Somatostatin levels during infancy, pregnancy, and lactation: A review. Peptides (N.Y.). 1995.
32. Watt H L, Kharmate G, Kumar U. Biology of somatostatin in breast cancer. Molecular and Cellular Endocrinology. 2008.
33. Suzuki H, Toyota M, Caraway H. Gabrielson E, Ohmura T, Fujikane T, et al. Frequent epigenetic inactivation of Wnt antagonist genes in breast cancer. British Journal of Cancer. 2008; 98.
34. Veeck J, Noetzel E, Bektas N, Jost E, Hartmann A, KnUchel R, et al. Promoter hypermethylation of the SFRP2 gene is a high-frequent alteration and tumor-specific epigenetic marker in human breast cancer. Molecular Cancer. 2008:7.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, the terms “process” and/or “processor” should be taken broadly to include a variety of electronic hardware and/or software based functions and components (and can alternatively be termed functional “modules” or “elements”). Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, as used herein various directional and dispositional terms such as “vertical” “horizontal”, “up”, “down”, “bottom”, “top”. “side”. “front”, “rear”. “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances of the system (e.g. 1-5 percent). Accordingly, this description is meant to be taken only by way of example, and not to otherwise limit the scope of this invention.

What is claimed is:

1. A method for use of a library for reference-based deconvolution of breast tissue and/or breast milk DNA methylation data assayed using a methylation process comprising:
accessing a library containing information related to an estimate of relative proportions of epithelial cells, endothelial cells, fibroblasts, adipocytes, B cells, CD4+ T cells, CD8+ T cells, NK cells, monocytes, and neutrophils associated with the breast tissue and/or breast milk; and
applying the library to diagnosis and treatment of a medical condition based upon patient information obtained from a sample of patient breast tissue and/or breast milk.

2. The method as set forth in claim 1, wherein the step of accessing includes operating a processor that provides information to a user interface based upon user requests thereto.

3. The method as set forth in claim 2, wherein the step of applying includes comparing the relative proportions of epithelial cells, endothelial cells, fibroblasts, adipocytes, B cells, CD4+ T cells, CD8+ T cells, NK cells, monocytes, and neutrophils associated with the breast tissue and/or breast milk in a patient being diagnosed to relative proportions of one or more of epithelial cells, endothelial cells, fibroblasts, adipocytes, B cells, CD4+ T cells, CD8+ T cells, NK cells, monocytes, and neutrophils associated with the breast tissue and/or breast milk in a disease-free subject.

4. The method as set forth in claim 3 wherein the methylation process comprises Illumina Infinium MethylationEPIC BeadChip.

5. The method as set forth in claim 4 wherein the processor is associated with a computing system having a network-based communication arrangement between a user, a storage site for the library and a server assembly that accesses the library and performs the step of comparing.

6. A non-transitory computer-readable medium for performing the steps of the method of claim 4.

7. A system for handling of a library for reference-based deconvolution of breast tissue and/or breast milk DNA methylation data provided in an assay based upon a methylation process comprising:
a processor assembly that is constructed and arranged to access a library containing information related to an estimate of relative proportions of epithelial cells, endothelial cells, fibroblasts, adipocytes, B cells, CD4+ T cells, CD8+ T cells, NK cells, monocytes, and neutrophils associated with the breast tissue and/or breast milk, and
wherein the processor assembly is constructed and arranged to apply the library to diagnosis and treatment of a medical condition based upon patient information obtained from a sample of patient breast tissue and/or breast milk.

8. The system as set forth in claim 7, wherein the processor provides information to a user interface based upon user requests thereto.

9. The system as set forth in claim 8, wherein the processor assembly is constructed and arranged to compare the relative proportions of epithelial cells, endothelial cells, fibroblasts, adipocytes, B cells, CD4+ T cells, CD8+ T cells, NK cells, monocytes, and neutrophils associated with the breast tissue and/or breast milk in a patient being diagnosed to relative proportions of one or more of epithelial cells, endothelial cells, fibroblasts, adipocytes, B cells, CD4+ T cells, CD8+ T cells, NK cells, monocytes, and neutrophils associated with the breast tissue and/or breast milk in a disease-free subject.

10. The system as set forth in claim 9 wherein the methylation process comprises Illumina Infinium MethylationEPIC BeadChip.

11. The system as set forth in claim 10 wherein the processor is associated with a computing system having a network-based communication arrangement between a user, a storage site for the library and a server assembly that accesses the library and performs the step of comparing.
